(19) United States
(12) Patent Application Publication
(10) Pub. No.: US 2016/0287536 A1
(43) Pub. Date: Oct. 6, 2016

(54) Composition

(71) Applicant: THE UNIVERSITY OF NOTTINGHAM, Nottingham (GB)

(72) Inventors: Vincent Wilson, Nottingham (GB); John Scholefield, Nottingham (GB); Varut Lohsiriwat, Nottingham (GB)

(73) Assignee: The University of Nottingham, Nottingham (GB)

(21) Appl. No.: 15/038,355

(22) PCT Filed: Nov. 21, 2014
(86) PCT No.: PCT/GB2014/053446

§ 371 Date: May 20, 2016

(30) Foreign Application Priority Data
Nov. 22, 2013 (GB) 1320675.0

(51) Int. Cl. A61K 31/165; A61K 45/06; A61K 9/00; A61K 31/4422

(52) U.S. Cl. CPC A61K 31/165; A61K 31/4422; A61K 45/06; A61K 9/0031

(57) ABSTRACT

The present invention relates to compositions comprising an alpha-adrenoceptor ligand for use in the treatment of benign anorectal conditions, in particular haemorrhoids. In addition to an alpha-adrenoceptor ligand, the compositions of the inventions may additionally include a calcium channel activator. Compositions with guanfacine and S(−)BayK8644 are preferred.

The present invention relates to a composition, in particular a topical composition, comprising an α-adrenoceptor ligand for use in the treatment of benign anorectal conditions.

Benign anorectal conditions are common conditions, most frequently treated by reducing the symptoms rather than addressing the underlying cause. Haemorrhoids are a very common anorectal condition defined as the symptomatic enlargement and distal displacement of the normal anal cushion. It affects millions of people around the world, and represents a major medical and socioeconomic problem. In the UK, the National Health Service in 2008 estimated that about 50% of people experienced haemorrhoids at some time in their life, especially in the elderly or during pregnancy. The most common symptom of haemorrhoids is rectal bleeding associated with bowel movement. The abnormal dilatation and distortion of the vascular channels, together with destructive changes in the supporting connective tissue within the anal cushions, is a recognised histological finding in haemorrhoidal disease. In most instances, haemorrhoids are treated conservatively, using many methods such as local anaesthetic agents delivered topically or by suppository; suppository-delivered anti-inflammatory drugs, fibre supplement, and lifestyle modification. An operation is indicated when non-operative approaches have failed or complications have occurred. Several surgical approaches for treating the haemorrhoids have been introduced including haemorrhoidectomy and haemorrhoidopexy, but postoperative pain is inevitable and recovery takes many weeks. Some of the surgical treatments may cause other morbidites such as anal stricture and incontinence.

There is therefore a need for improved treatment for anorectal conditions, and in particular, improved treatment for haemorrhoids.

According to a first aspect, the invention provides a composition comprising an α-adrenoceptor ligand for use in the treatment of benign anorectal conditions.

Preferably the ligand is an α₁ and/or an α₂ adrenoceptor ligand. Preferably the ligand is an α₂ adrenoceptor ligand.

The alpha-2 (α₂) adrenoceptor (also known as the α₂-adrenergic receptor) is a G protein-coupled receptor (GPCR) associated with the Gi heterotrimeric G-protein. Naturally, the alpha-2 (α₂) adrenoceptor binds both norepinephrine released by sympathetic postganglionic fibres and epinephrine (adrenaline) released by the adrenal medulla.

α₂-adrenoceptors linked to Gi-protein inhibit adenylate cyclase and thus reduces cAMP formation. Since myosin light chain kinase, an essential enzyme in the mechanism of muscular contraction, is inactivated by a cAMP-dependent protein kinase, the reduction of cAMP formation is associated with vasoconstriction. Therefore, the activation of α₂-adrenoceptors causes vasoconstriction. Like α₁-adrenoceptors, α₂-adrenoceptors also play an important role in the regulation of vascular tone.

In the autonomic nervous system, α₂-adrenoceptors located on the cell membrane of presynaptic neurons, sometimes known as autoreceptors, are responsible for the control of neurotransmitter release via a negative feedback pathway. Other functions of the α₂-adrenoceptor subtype include the contraction of sphincter in the gastrointestinal tract, the activation of platelet aggregation, and the inhibition of insulin and glucagon release from the pancreas.

The α₂-adrenoceptor ligand may be an agonist or an antagonist of the α₂-adrenoceptor. Agonists of the α₂-adrenoceptor include apraclonidine, brimonidine, clonidine, detomidine, dexmedetomidine, guanabenz, guanfacine, lofexidine, medetomidine, romifidine, tizanidine, tolonidine, xylazine, fadolmidine, xylometazoline and oxymetazoline or pharmaceutically active salts, esters, amides or N-oxides thereof. Antagonists of the α₂-adrenoceptor include atipamezole, cirazoline, efaroxan, idazoxan, mianserin, mirtazapine, napitane, phenoxybenzamine, phentolamine, rauwolscine, setiptiline, tolazoline and yohimbine or pharmaceutically active salts, esters, amides or N-oxides thereof. In a preferred embodiment the α₂-adrenoceptor ligand is the agonist guanfacine.

In a preferred embodiment the ligand is guanfacine or a pharmaceutically active salt, ester, amide or N-oxide thereof.

Salts of a compound are obtainable by reacting the compound with suitable acids and bases. The compounds can in one embodiment be used in the form of the corresponding salts with inorganic or organic acids or bases. Examples of such salts are alkali metal salts, in particular sodium and potassium salts, hydrochloride or ammonium salts. Specific examples of pharmaceutically acceptable salts are non-toxic inorganic or organic salts such as acetate, aconitate, ascorbate, benzoate cinnamate, citrate, embonate, formiate, fumarate, glutamate, glycolate, chloride, bromide, lactate, maleate, malonate, mandelate, methanesulfonate, naphtaline-2-sulfonate, nitrate, perchlorate, phosphate, phthalate, salicylate, sorbate, stearate, succinate, sulphate, tartrate, and toluene-p-sulfate. Such salts can be produced by methods known to the skilled reader and described in the prior art.

Compounds may also be provided in the form of their esters, their amides or their N-oxides. Such derivatives can be produced by methods known to the skilled reader and described in the prior art.

The compounds in the composition of the invention may also be provided as pro-drugs or any other bioprecursor which are converted in use into the active agents.

The composition of the invention may include more than one α-adrenoceptor ligand. The composition of the invention may include more than one α₂-adrenoceptor ligand.

In addition to an α-adrenoceptor ligand a composition for use in the invention may also include a calcium channel activator. The calcium channel activator may be a dihydropyridine-based calcium channel activator, such as S(−)-BayK8644 or a pharmaceutically active salt, ester, amide or N-oxide thereof.

In a preferred embodiment the composition comprises guanfacine and S(−)-BayK8644 or a pharmaceutically active salt, ester, amide or N-oxide thereof.

The composition may also comprise an inhibitor of nitric oxide synthase. In a preferred embodiment the inhibitor of nitric oxide is asymmetric dimethyl arginine (ADMA). Preferably the composition comprises an α₂-adrenoceptor agonist and ADMA.

The composition may further comprise additional agents, these may include one or more of the followings, a steroid (such as hydrocortisone or a pharmacologically acceptable derivative thereof), an analgesic agent, an antimicrobial agent, an antiviral agent, an antifungal agent, an anti-inflammatory agent and an antidiarrheal agent.

Benign anorectal conditions may include one or more of haemorrhoids, piles (the pathological condition of haemorrhoids), anal fissures, post operative conditions following haemorrhoidectomy, anal symptoms following vaginal delivery (with or without episiotmy), and anorectal vascular malformations

Preferably the anorectal condition is haemorrhoids and/or piles.

Preferably the composition is intended for topical administration. Preferably if a composition is administered topically it has only a local affect in the area of administration thus avoiding the side effects of some systemically administered compositions. For example, guanfacine is known if administered systemically or orally to have side effects such as dry mouth and dizziness, such side effects would be avoided by applying topically in the anorectal area.

The composition may be a topical composition in a form suitable for direct application to the colon, rectum, anorectum, perianal region or the anal canal. Suitable forms for topical administration include an enema, suppository, ointment, lotion, gel, foam, paste, cream, emollient, suspension, solution, oil, spray, powder or adhesive patch.

The composition for topical administration may also comprise skin penetrating agents, particularly a sulphoxide, such as dimethyl sulphoxide (DMSO), amides, pyrrolidones, organic solvents, laurocaprom and calcium thioglycollate, all of which are suitable skin penetrating agents.

A composition of the invention may be packaged in a unit dosage form, for example in the form of blister packs or sachets, each pack or sachet containing a unit dose of gel, cream or ointment etc. Alternatively the composition may be provided in a metered dosing device, for example a pump device for dosing a predetermined volume of a topical composition.

The preparation of compositions as described herein and examples of conventional additives are known to the skilled reader and are discussed in, for example, Remington's Pharmaceutical Sciences 16th edition, Osol, A. Ed. (1980).

A composition of the invention may be intended to be administered one or more time a day, for example, two or more times a day, three or more times a day, or perhaps more often.

A composition of the invention may comprise between about 0.03% and about 0.1% by weight of an α-adrenoceptor ligand, preferably an α₂-adrenoceptor ligand, more preferably guanfacine.

Alternatively a composition of the invention may be formulated and include instructions for use such that in use the composition comprises between about 0.03% and about 0.1% by weight of an α-adrenoceptor ligand, preferably an α₂-adrenoceptor ligand, more preferably guanfacine.

Whilst not wishing to be bound by any particular theory, a composition of the invention is believed to work by altering blood flow to the anorectal region. In particular, by causing arterial and/or venous constriction in the anal region, and in particular within haemorrhoidal tissue. The aim of the present invention is to treat the underlying cause of the anorectal condition, rather than just alleviate the symptoms. For example, current treatments for haemorrhoids relieve the symptoms by giving pain relief or anti-inflammatory drugs without actually addressing the cause of the condition. Preferably by targeting the α-adrenoceptors, and in particular the α₂-adrenoceptors, the composition of the invention can cause increased vasoconstriction in the anorectal region.

According to another aspect, the invention provides the use of an α-adrenoceptor ligand, or a pharmaceutically active salt, ester, amide or N-oxide thereof, in the manufacture of a medicament for the treatment of an anorectal condition. Preferably the medicament is a topical medicament.

According to a yet further aspect, the invention provide a topically acting pharmaceutical composition comprising an α-adrenoceptor ligand or a pharmaceutically active salt, ester, amide or N-oxide thereof, and a pharmaceutically acceptable carrier.

According to another aspect, the invention provides, a method of treatment of a benign anorectal condition in a subject comprising administering to the subject an effective amount of a composition comprising an α-adrenoceptor ligand or a pharmaceutically active salt, ester, amide or N-oxide thereof. Preferably the composition is administered topically.

The method may be carried out on a human or non-human animal subject.

According to a further aspect, the invention provides a composition comprising guanfacine, or a pharmaceutically active salt, ester, amide or N-oxide thereof, for use in the treatment of an anorectal condition. Preferably the anorectal condition is haemorrhoids and/or piles. Preferably the composition is for topical administration. The composition may also comprise a calcium channel activator, such as S(−)-BayK8644.

According to another aspect, the invention provides the use of guanfacine, or a pharmaceutically active salt, ester, amide or N-oxide thereof, in the manufacture of a medicament for the treatment of an anorectal condition. Preferably the medicament is a topical medicament. Preferably the anorectal condition is haemorrhoids and/or piles. The medicament may also comprise a calcium channel activator, such as S(−)-BayK8644.

According to another aspect, the invention provides, a method of treatment of a benign anorectal condition in a subject comprising administering to the subject an effective amount of a composition comprising guanfacine, or a pharmaceutically active salt, ester, amide or N-oxide thereof. Preferably the composition is administered topically. The composition may also comprise a calcium channel activator, such as S(−)-BayK8644. Preferably the anorectal condition is haemorrhoids and/or piles.

The skilled man will appreciate that all preferred aspects of the invention described with reference to only some aspects of the invention can be applied to all aspects of the invention.

The invention will be further described, by means of non-limiting example only, with reference to the following experimental examples and figures.

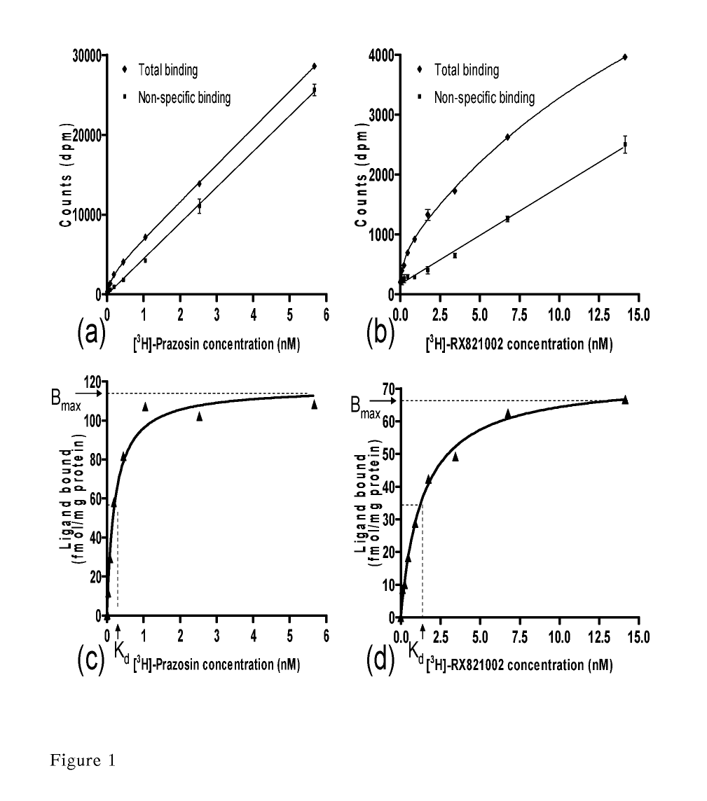

FIG. 1—illustrates saturation binding curves of (a) [³H]-prazosin and (b) [³H]-RX821002 to a membrane preparation of sheep rectal artery; total binding (TB, ♦) being defined as the binding in the absence of unlabelled specific receptor ligand, whereas non-specific binding (NSB, ▪) is defined as that remaining in the presence of 100 μM noradrenaline or 100 μM rauwolscine, respectively. Meanwhile, the curves of (c) [³H]-prazosin and (d) [³H]-RX821002 show the specific saturation binding (fmol/mg protein). Notably, specific binding (▴) is defined as the difference between binding in TB and NSB. Receptor density (Bₘₐₓ) and ligand dissociation constant (Kd) are respectively indicated with an arrow on the y-axis and x-axis of (c) and (d).

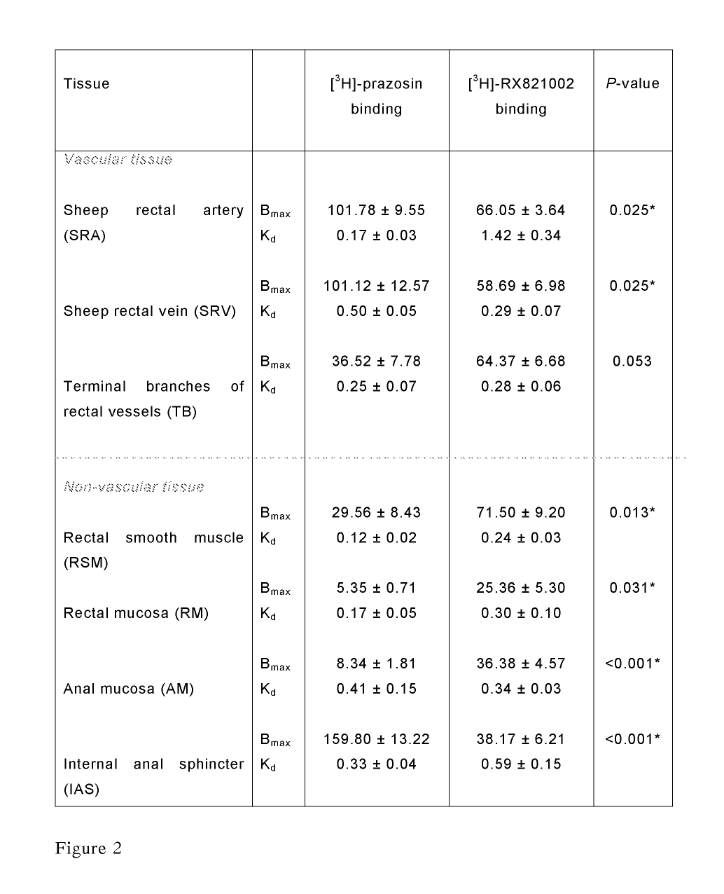

FIG. 2—illustrates receptor density (Bₘₐₓ-fmol/mg protein) and ligand dissociation constant (Kd-nM) values for [³H]-prazosin binding (α₁-adrenoceptor binding) and [³H]-RX821002 binding (α₂-adrenoceptor binding) in various sheep anorectal tissues. Results are given as mean±SEM of 3-6 observations. *P-value<0.05—significant difference in the density of α₁- and α₂-adrenoceptor binding sites (Student's unpaired t-test).

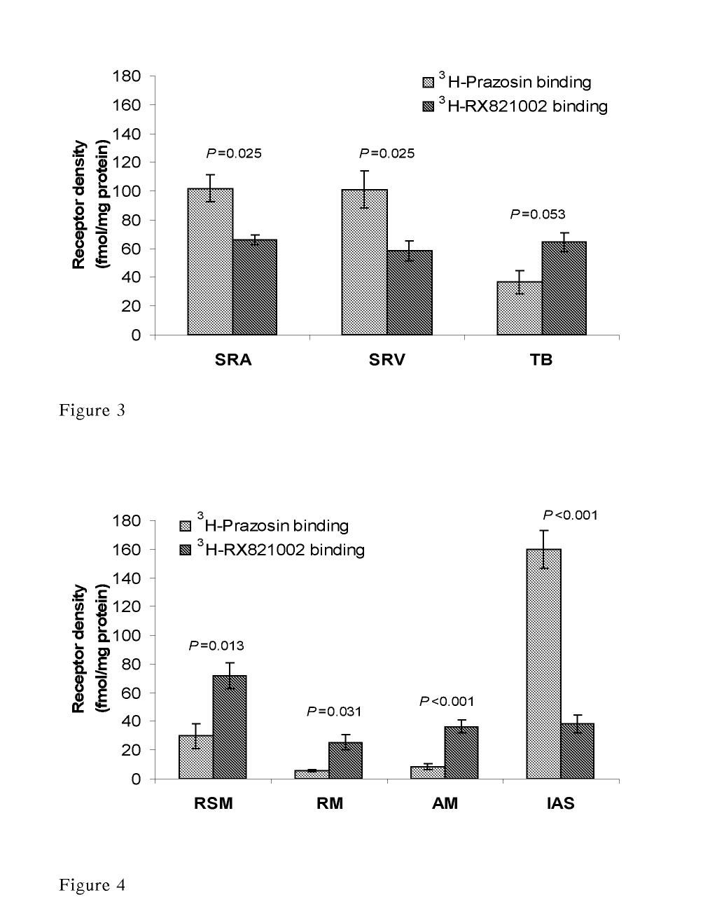

FIG. 3—illustrates receptor density (Bₘₐₓ-fmol/mg protein) of vascular structures of sheep anorectal tissues. Either student's unpaired t-test or Mann-Whitney U test was used to determine the difference of receptor density between [³H]-prazosin binding (α₁-adrenoceptor binding) and [³H]-RX821002 binding (α₂-adrenoceptor binding) in each type of the tissue. The vertical lines in the bar chart indicate the SEM of 3-4 observations. Abbreviations: SRA=sheep rectal artery, SRV=sheep rectal vein, and TB=the terminal branches of rectal vessels.

FIG. 4—Receptor density (Bₘₐₓ-fmol/mg protein) of non-vascular structures of sheep anorectal tissues. Either student's unpaired t-test or Mann-Whitney U test was used to determine the difference of receptor density between [³H]-prazosin binding (α₁-adrenoceptor binding) and [³H]-RX821002 binding (α₂-adrenoceptor binding) in each type of the tissue. The vertical lines in bar chart indicate the SEM of 3-6 observations. Abbreviations: RSM=rectal smooth muscle, RM=rectal mucosa, AM=anal mucosa, and IAS=internal anal sphincter.

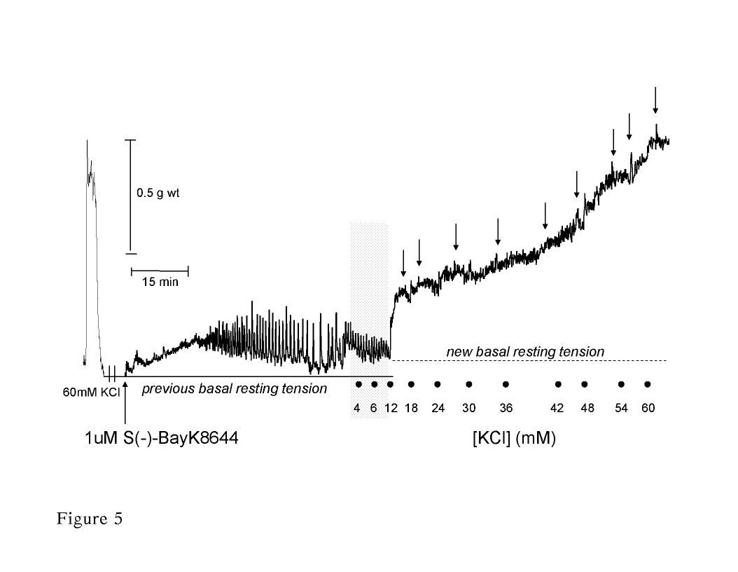

FIG. 5—A representative trace of the contractile response to KCl of the sheep isolated rectal vein in the presence of 1 μM S(−)-BayK8644. Spontaneous and periodic vascular contractions after the administration of S(−)-BayK8644, as well as a rise in basal resting tension, were noted. There is considered to be no contractile response to KCl at the concentration of 4 mM and 6 mM because there was no change in the frequency of contraction, although the baseline of vascular tone was slightly increased (shaded area). In contrast, at the KCl concentration of 12 mM (and thereafter), there was an increase in the frequency of contraction as well as a rise in the baseline of vascular tone. Therefore, the contractile responses to KCl were considered and measured against the new basal resting tension (dot line). Downward arrows (↓) indicate the maximum contraction of each KCl concentration.

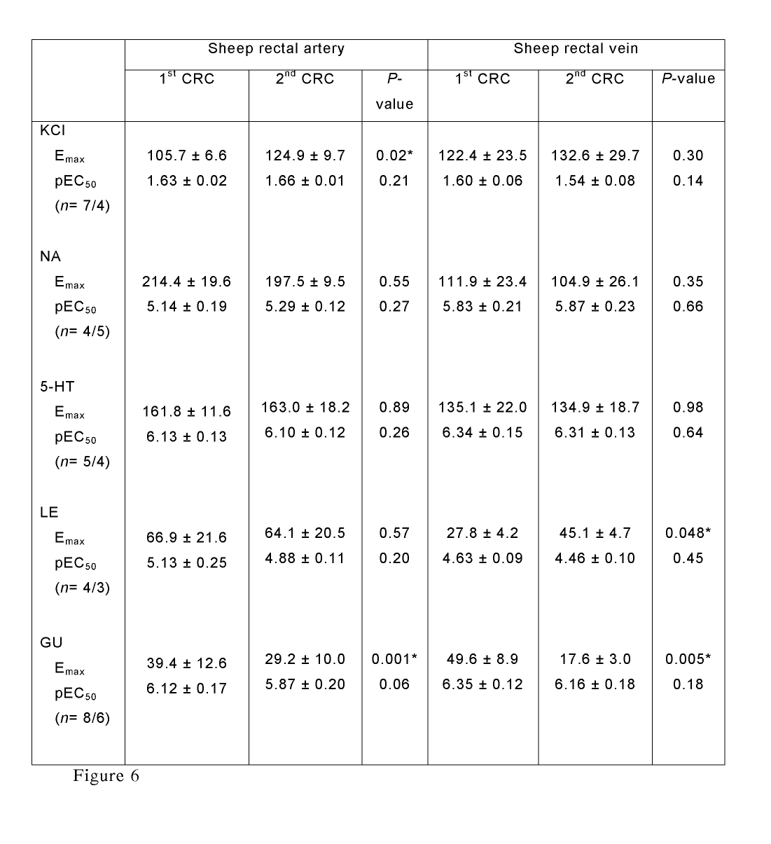

FIG. 6—illustrates the effect of KCl and various vasoconstrictors on the first and the second concentration-response curve (CRC) of isolated sheep rectal artery and vein. Maximum responses (Eₘₐₓ) are expressed as a percentage of the contraction to 60 mM KCl. Eₘₐₓ and pEC₅₀ values are shown as mean±SEM of 3-8 observations. The number of experiments performed in the sheep isolated rectal artery and vein is respectively given as n/n in the parentheses. * P-value<0.05 (Paired t-test), ** UK14304 produced a concentration-dependent contraction in 6 out of 12 veins, while the rest elicited no significant contraction (<10% of the response to 60 mM KCl). Abbreviations: NA=noradrenaline, 5-HT=5-hydroxytryptamine, LE=L-erythro methoxamine, GU=guanfacine, n/a=not applicable.

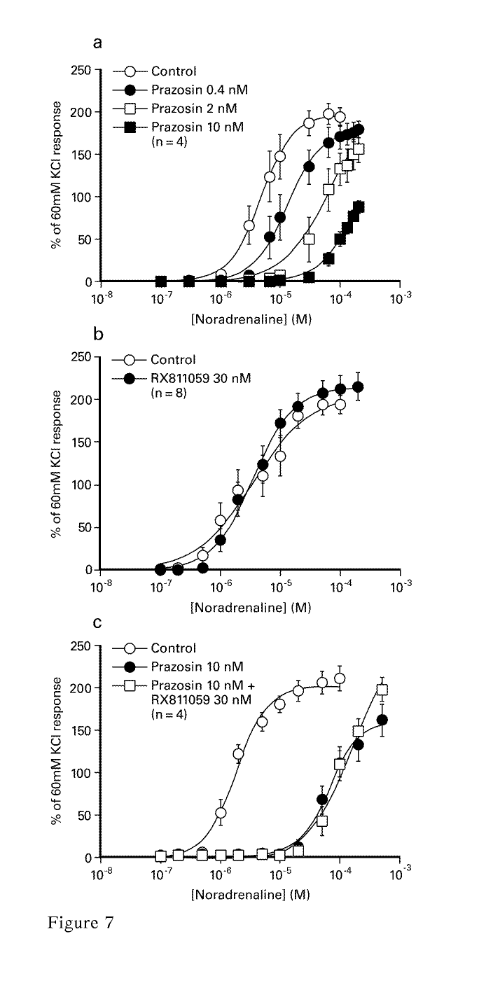

FIG. 7—illustrates the effect of (a) prazosin (PR), (b) RX811059 (RX), and (c) both antagonists in combination on noradrenaline-induced vascular contractions of sheep isolated rectal artery. Responses are expressed as a percentage of the contraction to 60 mM KCl, and are shown as mean±SEM of 4-8 observations.

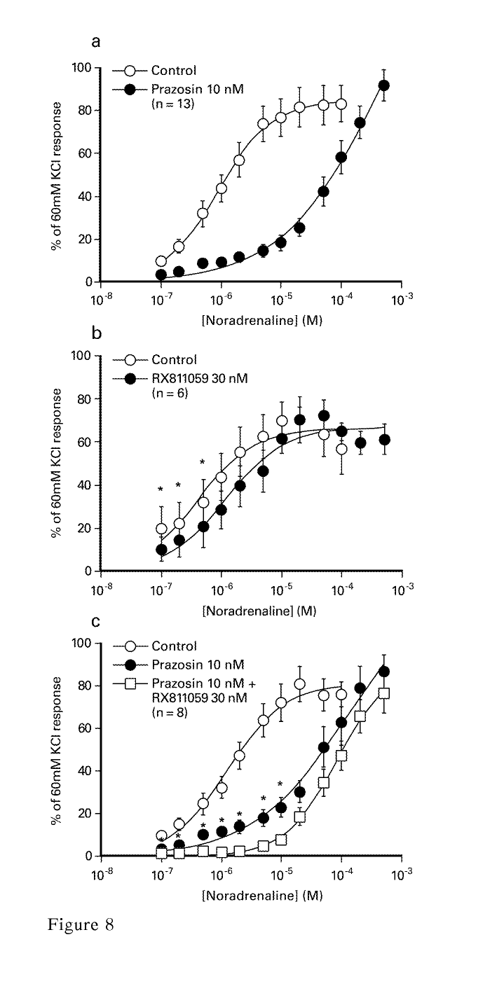

FIG. 8—illustrates the effect of (a) prazosin (PR), (b) RX811059 (RX), and (c) both antagonists in combination on noradrenaline-induced vascular contractions of sheep isolated rectal vein. Responses are expressed as a percentage of the contraction to 60 mM KCl, and are shown as mean±SEM of 4-13 observations. *P-value<0.05 between ∘ and • in (b), and between • and □ in (c).

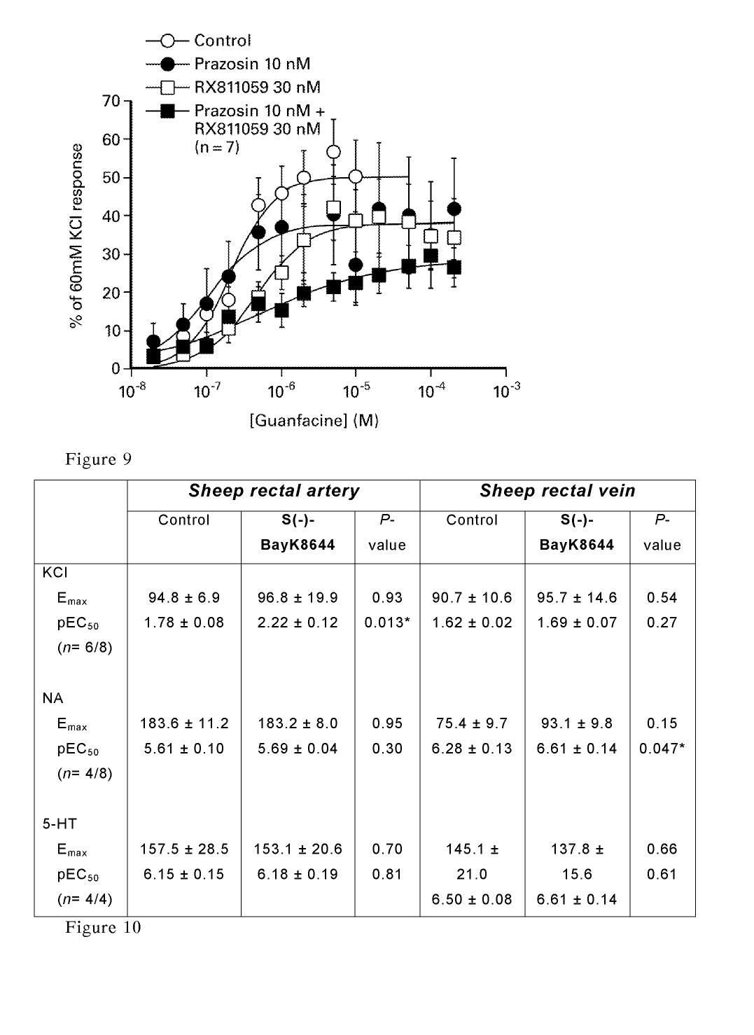

FIG. 9—illustrates the effect of (•) 10 nM prazosin, (□) 30 nM RX811059 and (▪) both antagonists in combination on guanfacine-induced contractions of sheep isolated rectal vein. The control concentration-response curve to guanfacine is represented by (∘). All points represent the mean of 7 observations and the vertical lines indicate the SEM.

FIG. 10—illustrates the effect of S(−)-BayK8644 (1 μM) on KCl, noradrenaline (NA) and 5-hydroxytryptamine (5-HT) mediated vasoconstriction on isolated sheep rectal artery and vein. Maximum responses (Eₘₐₓ) are expressed as a percentage of the contraction to 60 mM KCl. The Eₘₐₓ and pEC₅₀ values are shown as mean±SEM of 4-8 observations. The number of experiments performed on isolated sheep rectal artery and vein is respectively given as n/n in the parentheses.

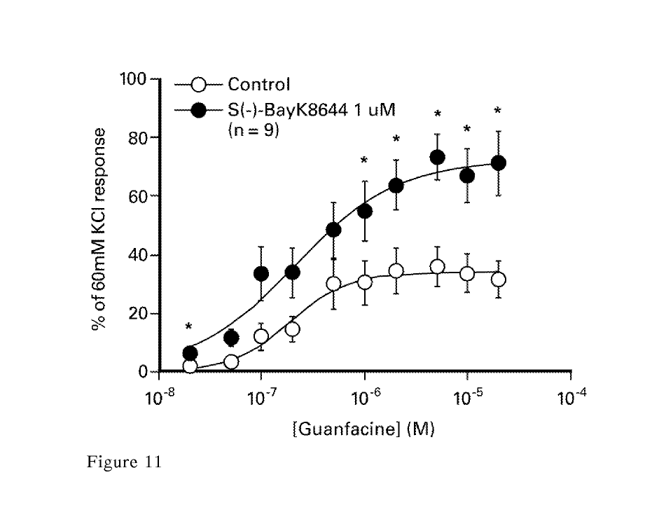

FIG. 11—illustrates the effect of (•) 1 μM S(−)-BayK8644 on guanfacine-induced contractions of sheep isolated rectal vein. The control concentration-response curve to guanfacine is represented by (∘). All points represent the mean of 9 observations and the vertical lines indicate the SEM. *P-value<0.05 (Unpaired t-test).

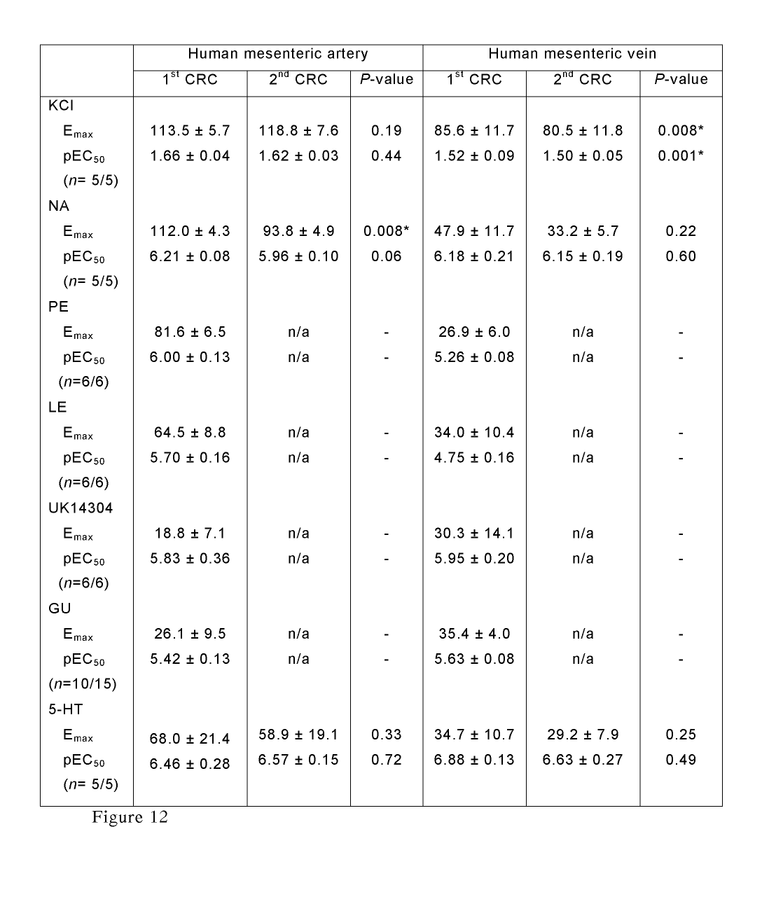

FIG. 12—illustrates the effect of KCl and various vasoconstrictors on the first and the second concentration-response curve (CRC) of the human isolated mesenteric (colonic/rectal) artery and vein. Maximum responses (Eₘₐₓ) are expressed as a percentage of the contraction to 60 mM KCl. The Eₘₐₓ and pEC₅₀ values are shown as mean±SEM of 3-15 observations. The number of experiments performed in the human mesenteric artery and vein is respectively given as n/n in the parentheses. * P-value<0.05, Abbreviations: NA=noradrenaline, PE=phenylephrine, LE=L-erythro methoxamine, GU=guanfacine, 5-HT=5-hydroxytryptamine, SMT=sumatriptan, ET-1=endothelin-1, n/a=not applicable.

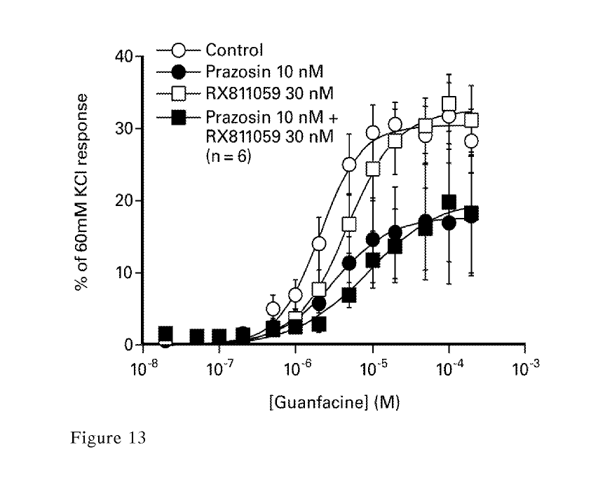

FIG. 13—illustrates the effect of (•) 10 nM prazosin, (□) 30 nM RX811059 and (▪) both antagonists in combination on guanfacine-induced contractions of human isolated mesenteric vein. The control concentration-response curve to guanfacine is represented by (∘). All points represent the mean of 6 observations and the vertical lines indicate the SEM.

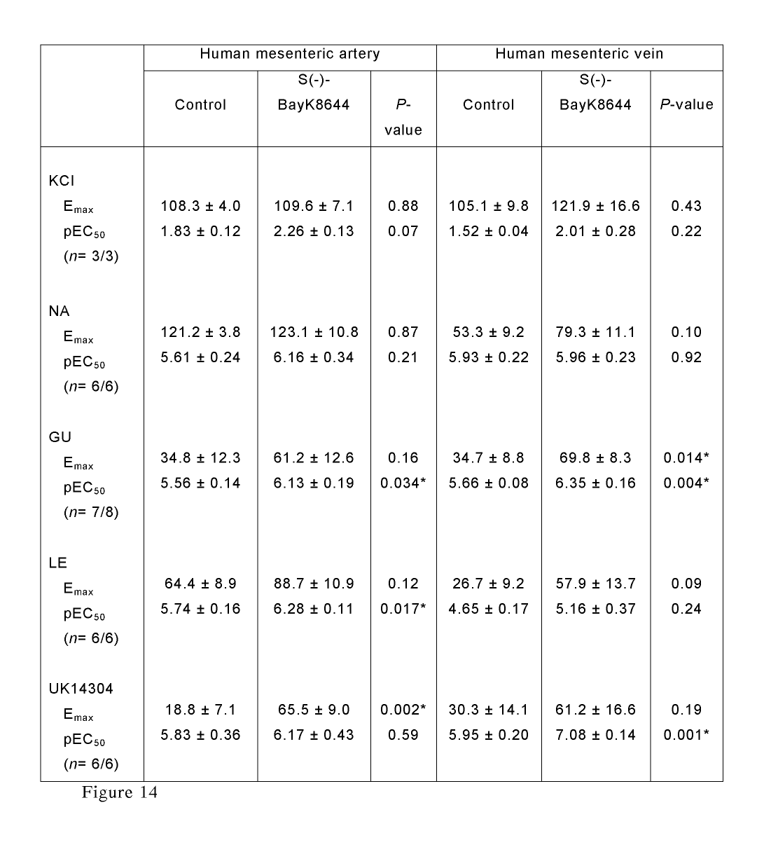

FIG. 14—illustrates the effect of S(−)-BayK8644 (1 μM) on vascular contractions of human isolated mesenteric (colonic/rectal) artery and vein induced by KCl, noradrenaline (NA), guanfacine (GU), L-erythro methoxamine (LE), and UK14304. Maximum responses (Eₘₐₓ) are expressed as a percentage of the contraction to 60 mM KCl. The Eₘₐₓ and pEC₅₀ values are shown as mean±SEM of 6-8 observations. The number of experiments performed in the human mesenteric artery and vein is respectively given as n/n in the parentheses. *P-value<0.05.

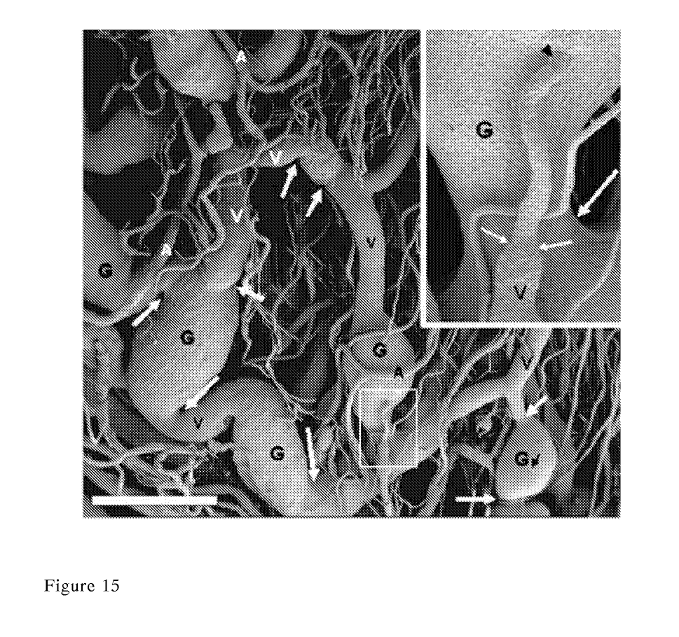

FIG. 15—illustrates an electron scanning micrograph of a microcast of a human anal cushion, showing arterioles (A), small venules (V) and dilated venous vessels (vascular glomerula, G). Note that the dilated vascular glomerula contributes the greatest volume to the structure and is associated with sphincter-like structures (see arrows) that are thought to be important in preventing stasis of blood.

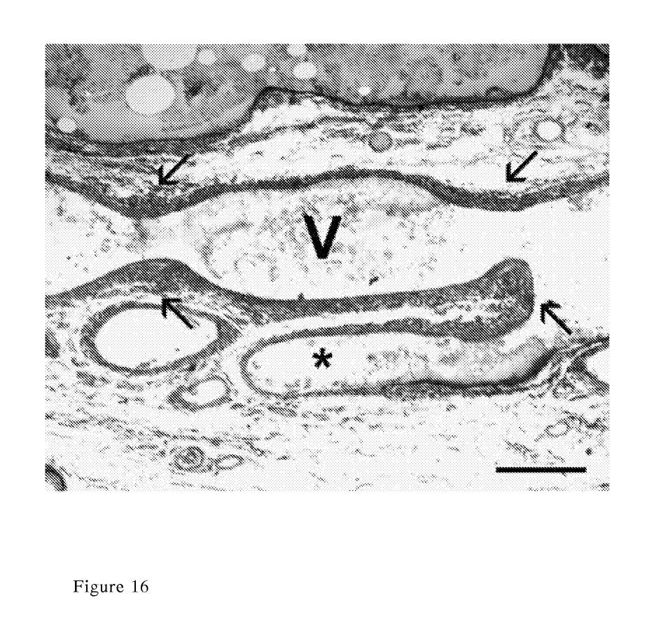

FIG. 16—illustrates azan staining of longitudinal segments of human haemorrhoids. (V) denotes the an example of a vein with the plexus and the arrows show areas of narrowing which is associated with a higher density of contractile protein (stained blue) (From Aigner at al., 2009 Int J Colorectal Dis 24: 105-113).

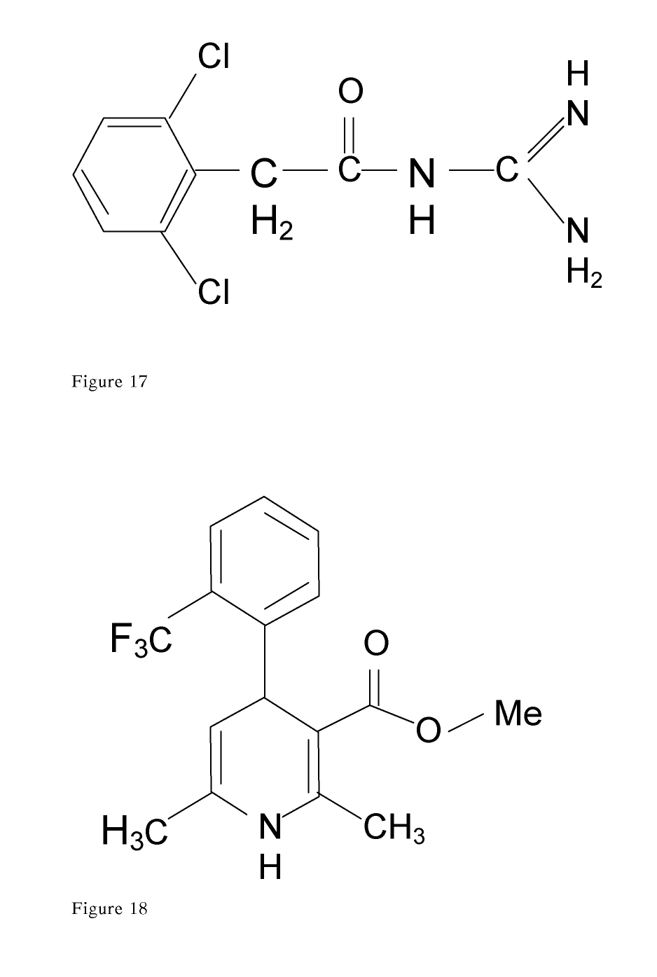

FIG. 17—illustrates the structure of guanfacine.

FIG. 18—illustrates the structure of S(−)-BayK8644.

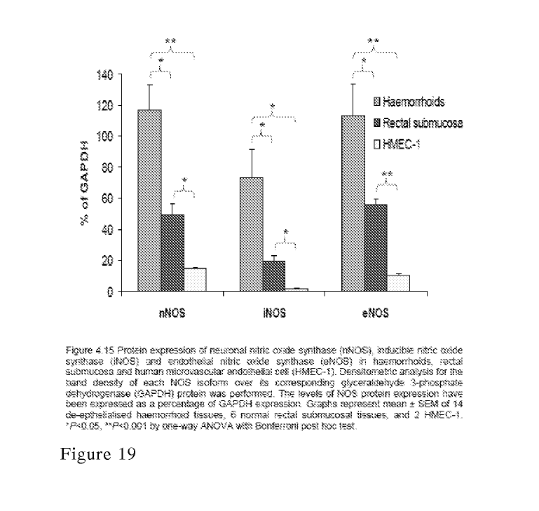

FIG. 19—illustrates the protein expression of neuronal nitric oxide synthase (nNOS), induced nitric oxide synthase (inNOS), and endothelial nitric oxide synthase (nNOS), in haemorrhoids, recatal submucosa and human microvascular endothelial cells (HMEC-1). Densitometric analysis for the band density of each NOS isoform over its corresponding GAPDH protein was performed. The levels of NOS protein expression are expressed as a percentage of GAPDH expression. The graphs represent mean±SEM of 14 de-epithelialised haemorrhoid tissues, 6 normal rectal submucosal tissues and 2 HMEC-1. *P-value<0.05, **P-value<0.001 by one way ANOVA with Bonferroni post hoc test.

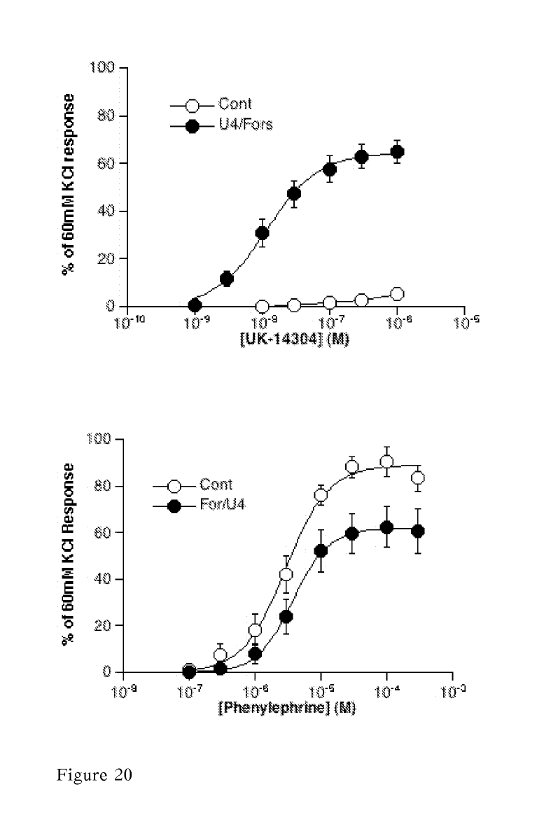

FIG. 20—illustrates a comparison of the effect of a combination of the vasoconstrictor U46610 (U4) and forskolin (For) against contractions to brimonidine (UK-14304) and phenylephrine in the porcine isolated tail artery. U46619 was added to produce a contraction equivalent to 60% of KCl followed by sufficient to forskolin to causes a relaxation back to baseline. Cumulative concentration response curves to brimondine and phenylephrine were elicited in the presence and absence of the combination of U4/For. Responses have been expressed a percent of the contraction to 60 mM KCl and represent the mean of 5-6 separate observations.

### 1. DENSITY OF α₂-ADRENOCEPTORS IN ANORECTAL VASCULAR AND NON-VASCULAR TISSUE

### Methods

### Tissue Preparation

Fresh sheep anorectal tissues were dissected from whole buttock from sheep within four hours of slaughter. The buttocks were delivered in a dry bag and stored at 4° C. until dissected. The tissues used in this experiment included rectal smooth muscle (RSM), rectal mucosa (RM), anal mucosa (AM), internal anal sphincter (IAS), sheep rectal artery (SRA), sheep rectal vein (SRV), and the terminal branches of rectal vessels (TB). The dissected tissues were kept at −20° C. until used. Prior to starting an experiment, the tissue was removed from the freezer and allowed to defrost on ice.

For the radioligand binding studies of α-adrenoceptors, approximately 3.8 grams of dissected tissue was cut into small pieces and homogenised in 10 volumes of ice-cold Tris buffer (50 mM; pH 7.6) using an Omni-macro homogeniser (OMNI International Ltd., USA). The tissue was homogenised at the rotational speed of 20,000 revolutions per minute, for periods of 20-30 seconds and then allowed to rest in an ice bucket for 10-20 seconds. This process was repeated until the tissue was completely homogenised. Next, the cell homogenate was centrifuged at 1,500 g for 10 minutes at 4° C. (SIGMA 3-18 centrifuge, SIGMA Laborzentrifugen GmbH, Germany). Supernatant was then filtered through a surgical gauze to remove unwanted non-homogenised tissue.

The cell homogenate was centrifuged again at 28,000 g for 30 minutes, 4° C. and the membrane pellet was rinsed with ice-cold Tris buffer and rehomogenised in 2 volumes of ice-cold Tris buffer using a Polytron blender (Ultra-Turrax TR50 homogeniser, Janke & Kunkel GmbH, Germany) at the rotational speed of 10,000 revolutions per minute for 15 seconds. The membrane preparation was kept in the ice until used.

The membrane preparation for each α-adrenoceptor radioligand binding assay consisted of a different number of sheep tissue: one for rectal smooth muscle (RSM), one for rectal mucosa (RM), 2-4 for anal mucosa (AM), 8-10 for internal anal sphincter (IAS), 20-28 for terminal branches of rectal arteries and veins (TB), and 16-20 for sheep rectal artery (SRA) and sheep rectal vein (SRV). The protein content of each membrane preparation was determined by the Lowry method (Lowry et al., 1951). Based on 3.8 grams of tissue used in α-adrenoceptor radioligand binding assay, the protein concentration (mg/ml) of each tissue was given as follows: 0.94±0.07 for RSM (n=10), 1.82±0.10 for RM (n=8), 2.22±0.10 for AM (n=9), 1.71±0.09 for IAS (n=7), 0.81±0.03 for TB (n=3), 0.66±0.08 for SRA (n=3), and 0.72±0.06 for SRA (n=4). The protein concentration of membrane preparation from the rat brain tissue, which was used for quality control of the [³H]-5-CT study, was about 1 mg/ml.

### Radioligand Binding Studies of α-Adrenoceptors

Radioligand binding assays (saturation assays) of α-adrenoceptors were performed in at least triplicate in each type of tissue. A total volume of the experimental aliquot was 500 μl, which was mixed in an LP4 tube. Non-specific binding was determined using an excessive amount of unlabelled specific receptor ligand.

### Saturation Assays of α-Adrenoceptors

Each assay consisted of 200 μl of membrane preparation, 200 μl of Tris buffer, 50 μl of radioligand, and 50 μl of unlabelled specific receptor ligand or buffer. The mixture was incubated at 25° C. for 30 and 60 minutes for α₁- and α₂-adrenoceptor studies, respectively. For the α₁-adrenoceptor study, [³H]-prazosin at a range of concentration between 0.01 nM and 5 nM was used and 100 μM noradrenaline was added as an unlabelled drug to determine non-specific binding. For the α₂-adrenoceptor study, [³H]-RX821002 at a range of concentration between 0.05 nM and 10 nM was used, and 100 μM rauwolscine was added to determine non-specific binding.

In order to terminate the aqueous reaction and separate the radioligand-receptor complex from unbound (free) radioligand, the assay mixture was rapidly filtered under negative pressure through filter paper in the Brandel Cell Harvester (Brandel Inc., USA) when the incubation period was complete. The harvesting methods are described as follows. First, about 10 minutes before the end of incubation time, a Brandel Cell Harvester was prepared by putting chilled water into the reservoir, connecting up all tubes, putting the lever into the harvesting mode, and then washing the system through with the chilled water. Next, chilled water in the reservoir was replaced with chilled 50 mM Tris-EDTA 1 mM buffer (pH 7.4). The through system with a first filter paper (Brandel GF/B fired filters, Brandel Inc, USA) was washed with chilled Tris-EDTA buffer. The assay mixture in each LP4 tube was then filtered under negative pressure through the filter paper. Each LP4 tube was washed and filtered 3 times with the chilled buffer. The radioligand-receptor complex was trapped on the filter paper whereas the unbound radioligand passed through the filter into a waste tank. Before harvesting the new mixture from LP4 tubes, a filter paper was replaced with a new filter paper and soaked with the buffer. The harvesting procedures were repeated until finished. Next, each filter containing radioligand-receptor complex was transferred to a 6 ml insert vial. Three ml of scintillation fluid (PerkinElmer, UK) was subsequently added into each vial, and left at least 8 hours (preferably overnight) before processing for radioactivity measurement. The amount of radioactivity bound to the filters was measured using a liquid scintillation analyser (Tri-Carb 2100TR, PerkinElmer, UK). The radioactivity was reported as disintegrations per minute (dpm).

### Analysis of Protein Concentration in Membrane Preparation

In order to calculate the amount of radioligand binding (fmol) per mg protein, protein content of each membrane preparation was measured by the Lowry method (Lowry et al., 1951 J Biol Chem, 193, 265-275), using bovine serum albumin (1 mg/ml; BSA) as a standard. Briefly, nine standard concentrations of 0-0.4 mg protein/ml were made by diluting BSA with distilled water in Bijoux tubes, with a total volume of 200 μl each. The membrane preparation was also diluted with distilled water in a Bijoux tube to make a 1:5, 1:10 and 1:20 dilution. Lowry AB solution was prepared by adding 20 ml of Lowry A solution to 100 μl 2% sodium potassium tartrate and 100 μl 1% CuSO4 (Lowry B solution). Next, all standard and sample tubes were added with 1 ml of the Lowry AB solution and left at room temperature for 10 minutes. Meanwhile, a 1:1 solution of Folin reagent to water was made up and 100 μl of this added into each tube after the 10-minute incubation was complete. The final mixture was then incubated at room temperature for 45 minutes (this can be left up to 3 hours) and the mixture was pipetted out into a clear 96 well plate at a volume of 200 μl/well. Protein concentration in each well was determined using an enzyme immunoassay plate reader (MRX and Revelation software version 4.22, Dynex, USA) fitted with a 750 nm filter. Protein concentration was calculated as mg/ml.

### Data and Statistical Analysis

Saturation radioligand binding data were analysed using non-linear curve fitting programme by Graphpad Prism 4.0 (Graphpad Software Inc., USA). Specific binding of the radioligand to the membranes was calculated by subtracting the non-specific binding from the total binding. Receptor density (Bₘₐₓ) and ligand dissociation constant (Kd) were then calculated using the specific saturation binding curve of each experiment.

The whole data set was compiled and compared using SPSS software (version 15.0 for Window, SPSS Inc., USA). The difference between the mean values of Bₘₐₓ and Kd obtained from each receptor was considered significant if P-value<0.05, using either Student's unpaired t-test, Mann-Whitney U test or one-way analysis of variance (ANOVA). Data are presented as mean±standard error of the mean (SEM).

### Radioligands and Drugs

Radioligands: [³H]-prazosin (3.22 TBq/mmol, GE Healthcare UK Ltd.), [³H]-RX821002 (2-(2-methoxy-1,4-benzodioxan-2-yl)-2-imidazoline) (2.37 TBq/mmol, GE Healthcare UK Ltd.), Drugs: ascorbic acid (BHD Laboratory Supplies, UK), calcium chloride (VWR International Ltd., UK), EDTA-ethylenediaminetetraacetic acid (BHD Laboratory Supplies, UK), folin reagent (Sigma-Aldrich, UK), 5-hydroxytrpytamine (Sigma-Aldrich, UK), pargyline hydrochloride (Sigma-Aldrich, UK), noradrenaline bitartrate (Sigma-Aldrich, UK), Rauwolscine hydrochloride (Carl Roth GmbH, Germany), Tris (hydroxymethyl) methylamine (VWR International Ltd., UK)

### Results

### Saturation Study of α-Adrenoceptors

Saturation assays of [³H]-prazosin and [³H]-RX821002 binding yielded a monophasic saturation isotherm in all sheep anorectal tissues. An example of binding isotherms is shown in FIG. 1, which illustrates an individual saturation isotherm of [³H]-prazosin and [³H]-RX821002 to the membrane of sheep rectal arteries. Similar results were obtained in 25 experiments of [³H]-prazosin (5 for anal mucosa, 4 for rectal smooth muscle/rectal mucosa, and 3 for internal anal sphincter/sheep rectal vessels and their terminal branches) and 28 experiments of [³H]-RX821002 (6 for rectal smooth muscle, 4 for sheep rectal veins/internal anal sphincter/rectal and anal mucosa, and 3 for sheep rectal arteries/the terminal branches of rectal vessels).

The non-specific binding of each radioligand determined in the presence of an excess of unlabelled noradrenaline for [³H]-prazosin and rauwolscine for [³H]-RX821002 was linearly correlated to the radioligand concentration. Collectively, in all sheep anorectal tissues, non-specific binding (NSB) varied from 15-90% of total [³H]-prazosin binding (α₁-adrenoceptor binding), and 7-47% of total [³H]-RX811002 binding (α₂-adrenoceptor binding) at levels corresponding to their respective Kd. Notably, NSB of [³H]-prazosin was remarkably high in the rectal and anal mucosa, whereas that of [³H]-RX821002 was highest in the sheep rectal artery and vein.

Specific binding for [³H]-prazosin and [³H]-RX821002 was noted in both vascular and non-vascular structures of sheep anorectal tissues. Regarding the receptor density (Bₘₐₓ), rectal smooth muscle and anorectal mucosa had a significantly higher Bₘₐₓ for [³H]-RX821002 binding compared to that of [³H]-prazosin binding. Meanwhile, the Bₘₐₓ of [³H]-prazosin binding was significantly greater in the internal anal sphincter, rectal arteries and veins (FIG. 2).

Although the Bₘₐₓ of [³H]-prazosin binding was significantly higher than that of [³H]-RX821002 binding in the rectal arteries and veins, this was not the case in the smaller vessels (FIG. 3). The [³H]-RX821002 binding found in all non-vascular tissues varied 2-3 folds, but [³H]-prazosin binding was practically absent in mucosal tissue (FIG. 4).

As shown in FIG. 2, the order of potency based on Kd value for [³H]-prazosin binding in sheep anorectal tissue was RSM>SRA=RM>TB>IAS>AM>SRV, and that for [³H]-RX821002 binding was RSM=RM=TB=SRV>AM>IAS>SRA. Focusing on vascular tissues, there was 2-3 fold difference in the Kd for [³H]-prazosin binding between artery and vein. Meanwhile, the difference in the Kd for [³H]-RX821002 binding between artery and vein was about 5 fold.

### Discussion

The present study revealed that sheep anorectal tissue contained both α₁-adrenoceptors ([³H]-prazosin binding sites) and α₂-adrenoceptors ([³H]-RX821002 binding sites). Among all sheep anorectal tissues, the internal anal sphincter had the highest density of α₁-adrenoceptor binding whereas anal and rectal mucosa had the lowest density of α₁-adrenoceptor binding. Meanwhile, rectal smooth muscle contained the highest density of α₂-adrenoceptor binding. The density of α₁-adrenoceptors was significantly higher than that of α₂-adrenoceptors in the rectal arteries and veins, but this was not the case in the smaller vessels. These tissues are dealt with separately below:

α₁- and α₂-Adrenoceptors

### Internal Anal Sphincter (IAS)

The characterisation of α₁-adrenoceptor in the IAS has been extensively studied in animals, especially in sheep and pig, as a model for developing new pharmacological treatment of faecal incontinence in man. Rayment and colleagues recently demonstrated that in sheep IAS the density of α₁-adrenoceptor binding sites was approximately 3-fold greater than that of α₂-adrenoceptor binding sites (Rayment et al., 2010 Br J Pharmacol, in press). These findings are comparable to the present study, in which Bₘₐₓ of α₁-adrenoceptor binding sites was 4 times higher than that of α₂-adrenoceptor binding sites.

With respect to the location of α₁- and α₂-adrenoceptor binding sites in sheep smooth muscle tissue, it is worth noting that the IAS, as well as rectal smooth muscle, is supplied by enteric neurons and their adrenergic nerve fibres. Hence, α₁- and α₂-adrenoceptor binding sites could be located in smooth muscle cells, inherent ganglionic cells and adrenergic nerves. This conclusion is supported by recent observations in sheep IAS where electric field stimulation and autoradiographic studies revealed the presence of α₁- and α₂-adrenoceptors on the smooth muscle bundles, and immunohistochemical analysis confirmed adrenergic innervation within the sheep IAS (Acheson et al., 2009 Neurogastroenterol Motil, 21, 335-345; Rayment et al., 2010 Br J Pharmacol, in press).

### Rectal Smooth Muscle

Unlike the IAS, sheep rectal smooth muscle possessed a significantly higher number of α₂-adrenoceptor binding than α₁-adrenoceptor binding.

### Anorectal Mucosa

Regarding the density of α-adrenoceptor binding sites in sheep anorectal mucosa, the density of α₂-adrenoceptor binding was approximately 5 times higher than that of α₁-adrenoceptor binding.

### Blood Vessels

With regards to the sheep vasculature, the density of α₂-adrenoceptors bound to [³H]-RX821002 was similar across three vessels (sheep rectal artery, sheep rectal vein, and their terminal branches); about 60 fmol/mg protein. However, the density of α1-adrenoceptors ([³H]-prazosin binding) was greatly reduced in small vessels; from about 100 fmol/mg protein in sheep rectal artery and vein to only 36.5 fmol/mg protein in their terminal branches (see FIG. 3 and FIG. 2 for more details).

The Kd values of radioligand for α₁- and α₂-adrenoceptor in sheep anorectal vessels were between 0.17 nM and 0.50 nM, except that of α₂-adrenoceptor in sheep rectal arteries—which was surprisingly high (1.42 nM). The difference in Kd values of α₂-adrenoceptor between sheep rectal artery and vein could account for the different contractile responses to α-adrenoceptor agonist/antagonist. The Kd values of α1-adrenoceptors bound to [³H]-prazosin in sheep anorectal vessels were fairly comparable to those from the study of various porcine vessels (thoracic aorta, palmar common digital artery, palmar lateral vein, splenic artery, ear artery and vein) by Wright and colleague, which ranged from 0.13 nM to 0.20 nM (Wright et al., 1995 Br J Pharmacol, 114, 678-688). However, the Kd values of α₂-adrenoceptor in sheep rectal vein and terminal branches of sheep rectal vessels (about 0.3 nM) were much lower than those of porcine vessels, which varied between 1.3-2.2 nM. It would appear that sheep rectal vessels and isolated porcine vessels had the similar property of α1-adrenoceptor bound to [³H]-prazosin, but α₂-adrenoceptor in sheep rectal vein and terminal branches of sheep rectal vessels, not in sheep rectal artery, had a high affinity for [³H]-RX821002 than isolated porcine vessels.

In summary, both receptors are located on sheep anorectal tissue with α₂-adrenoceptors showing a wider and more even distribution than α₁-adrenoceptors. The latter seems to be more preferentially located on the IAS.

### General Discussion

[³H]-prazosin, an α₁-adrenoceptor antagonist, and [³H]-RX821002, an α₂-adrenoceptor antagonist, were used as a subtype nonselective radioligand that has nearly equal affinity for the subtypes of each α₁- and α₂-adrenoceptor, respectively (Bylund & Toews, 1993 Am J Physiol, 265, L421-429).

Although the precise distribution of α-adrenoceptors cannot be determined by using radioligand binding studies, the density of such receptors and their affinity to a relevant radioligand provide very useful information. These could allow further studies on the location and functional properties of such receptors in sheep anorectal and human haemorrhoidal tissue, as well as in the mesenteric blood vessels. The further experimental studies include autoradiography and isolated vascular contractile study (wire myography)—the results of which are presented below.

The differences in the distribution of α₁- and α₂-adrenoceptors and 5-HT1B/1D receptors in the sheep anorectal tissue indicate that these receptors can be selectively targeted to treat anorectal conditions in man. For example, an α₂-adrenoceptor agonist may be a target for potential topical treatment for haemorrhoids because there was a higher density of α₂-adrenoceptors than α₁-adrenoceptors in the anorectal mucosa and in the small rectal vessels, whereas the underlying anal sphincter contained fewer density of α₂-adrenoceptor. Moreover, α₂-adrenoceptors do not have a major role in the sphincter contraction (Rayment et al., 2010 in press). Thus, treating haemorrhoids which are located in the mucosal/submucosal layer of the anal canal by topical administration of an α₂-adrenoceptor agonist would not greatly interfere with the function of underlying anal sphincter.

### 2. EXAMINATION OF THE PHARMACOLOGICAL CHARACTERISTICS OF α-ADRENOCEPTORS IN SHEEP AND ISOLATED RECTAL ARTERY AND VEIN

### Materials and Methods

### Wire Myography

Sheep mesenteric tissue, from the distal sigmoid colon to the anus, was obtained from a local abattoir. The sheep tissue was delivered in a dry bag and stored at 4° C. until dissected. The bundle of sheep rectal vessels was dissected from its mesentery within four hours of arrival from the abattoir. The vascular bundle was then kept in the pre-oxygenated Krebs-Henseleit buffer solution at 4° C. until used (on the same day as a preferential method or on the following day after storage overnight). No experiments were conducted on tissue stored for more than 24 hours after dissection. The Krebs solution consisted of 118.4 mM NaCl, 4.7 mM KCl, 1.2 mM MgSO₄, 1.2 mM KH₂PO₄, 1.25 mM CaCl₂, 24.9 mM NaHCO₃, and 11.1 mM glucose.

Before setting a wire myography experiment, the remaining loose connective tissue was meticulously removed from the rectal vessels. The internal diameter of sheep isolated rectal artery and vein was 450-650 μm and 500-1000 μm, respectively. Each 5-mm ring segment of the ‘clean’ vessels was then suspended between two 200-μm wire supports in an organ bath containing 20 ml Krebs-Henseleit buffer solution. The upper supporting wire was attached to an isometric force transducer, while the lower wire was fixed steadily to a clamped glass rod. The bath solution was maintained at 37° C., and continuously gassed with 95% O₂ and 5% CO₂. Isometric tension was continuously recorded using a PowerLab 4/25 via QUAD Bridge amplifier (ADInstruments, Inc, Australia).

After a 10-minute equilibration period, an initial tension of 8 gram weight (g wt) was gradually applied for an arterial segment and left to relax for 30 minutes. The final tension of SRA varied between 1.0 g wt and 2.8 g wt. For a venous segment, an initial tension of 1 g wt was applied and, 10 minutes later, the resting tension was finally re-adjusted to reach a tension of 0.5 g wt. Then the vein was left to relax for further 20 minutes. The final tension of SRV ranged from 0.08 to 0.25 g wt. Once the vascular segments were fully settled, the tissues were tested for contractile activity with 60 mM KCl on three occasions. The vascular contraction was allowed to reach maximum, which generally took about 1-4 minutes. Each KCl challenge was followed by two washouts of 37° C. Krebs solution and the tissues were allowed to rest for 20 minutes between the challenges. The maximum contraction (tension) produced by the third administration of 60 mM KCl was used as a reference contraction to which subsequent responses were compared. After the final (3rd) KCl challenge, the tissues were washed and left for 60 minutes before α-adrenoceptor agonist was administrated. To construct an agonist concentration-response curves (CRC), 0.3-0.4 log 10 increments of agonist solution was added e.g. at the agonist concentration of 100 nM, 200 nM, 500 nM, 1 μM, 2 μM, 5 μM, and so on. Each response was allowed to reach equilibrium or plateau before the next dose of agonist was added. The maximal response to the agonist was considered when no further increase in response occurred upon the addition of two or more consecutive doses of agonist. Notably, when the response of the tissue failed to achieve a maximum with an agonist concentration of 500 μM, the final response at this concentration was used to calculate to the maximum response.

In the case of the noradrenaline study, 10 μM cocaine was added into the organ bath 40 minutes prior to the administration of noradrenaline for inhibiting the uptake of noradrenaline by neural tissue within the vascular wall (re-uptake 1) (Westfall & Westfall, 2006 In Goodman & Gilman's the Pharmacological Basis of Therapeutics. eds Brunton, L. L., Lazo, J. S. & Paker, K. L. pp. 137-181. New York: McGraw Hill). Regarding KCl-induced vasoconstriction, the CRC was generated by the contractile response to 6 mM increments of KCl solution, starting from 0 mM to 60 mM.

Consecutive CRCs were generated to each agonist, separated by 60 minutes, and if there was no difference in the maximum response (Eₘₐₓ) and pEC₅₀ (defined as the negative logarithm of the concentration required to produce 50% of the maximum response) then a paired CRC protocol was used throughout. If, however, the CRCs were not reproducible then a single CRC, paired segment approach was adopted. The maximum response of each agonist concentration was recorded and used for the generation of the agonist CRC. If the response was either phasic or non-sustained, the highest point of the contractile tension would be used as the maximum response of that agonist concentration. If the vessel had spontaneous and periodic contraction after the administration of S(−)-BayK8644—which occurred in about 40% of the experiments on the SRV, the effect of agonist would be considered when there was an increase in the frequency of contraction as well as a rise in the baseline of vascular tone. FIG. 5 shows an example trace of venous contractile response to KCl in the presence of S(−)-BayK8644 and how to measure the contractile response when S(−)-BayK8644-induced spontaneous contraction occurred.

Selective α₁- and α₂-adrenoceptor antagonists used in this study were prazosin (Hoffman et al., 1979 Life Sci, 24, 1739-1745) and RX811059 (Harris & Clarke, 1993 Eur J Pharmacol, 237, 323-328), respectively. The α-adrenoceptor antagonists, flavonoids, or Ca²⁺ channel activators were added into the organ bath 40-50 minutes prior to the administration of α-adrenoceptor agonist.

### Data Analysis

Contractile responses of vascular smooth muscle to an agonist progressively extend as the concentration of such an agent is increased until the maximum response is achieved. The relation between the agonist concentration and response is generally described by a hyperbolic curve, based on the equation below, which is a modification of the Langmuir equation:

$$\frac{E}{{E}_{max}} = \frac{[ A ]}{[ A ] + {EC}_{50}} = \frac{[ A * R ]}{[ {R}_{0} ]}$$

Where [A]=concentration of agonist, [A*R]=concentration of agonist-receptor complex, [R₀]=concentration of total receptors, E=the response observed at concentration [A], Eₘₐₓ=the maximum response possible in the system, and EC₅₀=the concentration of agonist provoking 50% of the maximum response.

With respect to the potency of an agonist, the potency is a measure of the dilution in which it produces a specific response. A highly potent agonist causes a larger response at low concentrations. The potency is influenced by both the agonist's affinity (the ability of the agonist to bind to its receptor) and the agonist's efficacy (the ability of the agonist to cause a response once the receptor is bound). The commonest point of specific response used in determination of agonist potencies, as used in the present study, is the 50% of the maximum response. Thus, the potency of agonists is inversely proportional to EC₅₀ and is independent of the Eₘₐₓ. Since the EC₅₀ of agonists is usually given in the unit of ‘μmol/l’, a simple way to determine and compare agonist potencies is to express the potency using ‘pEC₅₀’ which is defined as the negative log 10 of the molar EC₅₀. The higher EC₅₀ is, the more potent the agonist is. For example, if the EC₅₀ of agonist X is 1 μmol/l and agonist Y is 10 μmol/l, the pEC₅₀ of X and Y are 6 and 5, respectively. Therefore, agonist X is more potent than agonist Y.

### Statistical Analysis

Maximum responses (Eₘₐₓ) are expressed as a percentage of the 3rd 60 mM KCl response. Agonist potency is expressed as pEC₅₀ (the negative logarithm of the concentration required to produce 50% of the maximum response). The Emax and pEC₅₀ values were determined using the KaleidaGraph curve-fitting programme (Synergy Software, Reading, Pa.). Results are expressed as mean±SEM of n observations, where n is the number of studies in tissue from different sheep.

All data were prepared and compiled using the SPSS® software (version 15.0 for Windows, Illinois, USA). The Kolmogorov-Smirnov test was used to test for the pattern of data distribution. Unpaired or paired Student's t-test was used to compare data between two groups when the data were in a normal distribution pattern. The Mann-Whitney U-test or Wilcoxon Signed Rank test was used to compare data between two groups when the data were in a non-normal distribution. If there were more than two groups being analysed, the ANOVA with appropriate post hoc test (Bonferroni or Dunnett's) would be used. A P-value<0.05 was considered statistically significant.

### Drugs

The drugs used were: S(−)-BayK8644 or (4S)-1,4-dihydro-2,6-dimethyl-5-nitro-4-[2-(trifluoromethyl)-phenyl]-3-pyridine carboxylic acid methyl ester (Tocris Bioscience, UK), R(+)-BayK8644 or (4R)-1,4-dihydro-2,6-dimethyl-5-nitro-4-[2-(trifluoromethyl)-phenyl]-3-pyridine carboxylic acid methyl ester (Tocris Bioscience, UK), cocaine hydrochloride (Sigma-Aldrich, UK), diosmin or 3′,5,7-trihydroxy-4′-methoxyflavone 7-rutinoside (Enzo Life Sciences, UK), L-erythro methoxamine (Norgine International, UK), guanfacine hydrochloride (Sigma-Aldrich, UK), 5-hydroxytryptamine (Sigma-Aldrich, UK), myricetin or 3,3′,4′,5,5′,7-hesahydroxyflavone (Sigma-Aldrich, UK), noradrenaline bitartrate (Sigma-Aldrich, UK), prazosin hydrochloride (Pfizer, UK), RX811059 or 2-(2-ethoxy-1,4-benzodioxan-2-yl)-2-imidazoline (Reckitt & Coleman, UK), sumatriptan succinate (Sigma-Aldrich, UK). All drugs used were of analytical grade.

All drugs were prepared in distilled water except noradrenaline, flavonoids, BayK8644 compounds. Noradrenaline was dissolved in 20 μM ethylenediaminetetraacetic acid (EDTA) aqueous solution to prevent catecholamine degradation. Diosmin, myricetin, S(−)-BayK8644 and R(+)-BayK8644 compounds were dissolved in dimethyl sulfoxide (DMSO, Sigma-Aldrich, UK) to prepare 0.01M stock solution. The volume of drug solution added into an organ bath was between 6-20 μl; therefore, the concentration of the vehicle in an organ bath never exceeded 0.1% v/v. Furthermore, the addition of DMSO or EDTA solution did not cause any change in vascular tension (unpublished observations). The drug concentrations reported in the following sections were the calculated final concentrations in the organ bath solution.

### Results and Conclusions

The data presented herein shows that sheep rectal vessels, and their terminal branches, possess α₁- and α₂-adrenoceptor binding sites. These two α-adrenoceptor subtypes play an important role in the regulation of vascular tone and blood flow in both systemic and regional circulation, including the mesenteric vascular bed. Thus demonstrating that these receptors may serve as a therapeutic target in the treatment of haemorrhoids and other anorectal conditions.

The data below illustrates the characteristics of α₁- and α₂-adrenoceptors on the sheep isolated rectal artery and vein on the basis of their responses to selective and non-selective α-adrenoceptor agonists/antagonists.

### The Effect of KCl and Various Vasoconstrictors on the SRA and SRV, and the Reproducibility of Concentration-Response Curves

In these experiments the following compounds were considered, noradrenaline the natural ligand for an α-adrenoceptor, 5-HT, L-erythromethoxamine a selective agonist of the α₁-adrenoceptor, and guanfacine a selective agonist of the α₂-adrenoceptor.

Exposure to 60 mM KCl caused a contraction of both SRA and SRV. The mean contraction to 60 mM KCl in the SRA was 3.14±0.09 g wt (n=119, range 0.54-6.16), and that in the SRV was 1.18±0.05 g wt (n=128, range 0.30-2.61). KCl caused a sustained concentration-dependent contraction of the SRA. Noradrenaline, 5-HT, L-erythro methoxamine and guanfacine also caused concentration-dependent contractions of the SRA, but the responses to noradrenaline and 5-HT were markedly greater than those to KCl and characterised by a rapid increase in tension that was not sustained (FIG. 6). In contrast, while L-erythro methoxamine and guanfacine also produced concentration-dependent contraction, the maximum effect was significantly less than that produced by KCl (FIG. 6). The contractions to guanfacine were slow to develop and sustained.

In the SRV KCl produced concentration-dependent contractions. Noradrenaline, 5-HT, L-erythro methoxamine, guanfacine and UK14304 also caused concentration-dependent contractions of the SRV, but the responses to L-erythro methoxamine, guanfacine and UK14304 were clearly lesser than those to KCl (FIG. 6). Meanwhile, the maximum responses to KCl, noradrenaline and 5-HT were fairly similar in the SRV (FIG. 6). Similar to those of the SRA, the responses to α-adrenoceptor agonists in the SRV were characterised by a greatly rapid increase in tension that was unsustained.

The relative order of agonist potency in the SRA was guanfacine=5-HT>noradrenaline>L-erythro methoxamine (FIG. 6). Similar to the SRA, the order of agonist potency in the SRV was guanfacine=5-HT>noradrenaline>L-erythro methoxamine (FIG. 6). Meanwhile, the relative order of maximum response from the first CRC, which was expressed as a percentage of the contraction to 60 mM KCl, in the SRA was noradrenaline>5-HT>L-erythro methoxamine>guanfacine, and that in the SRV was 5-HT>noradrenaline>guanfacine>L-erythro methoxamine (FIG. 6).

It was noted that guanfacine had the highest potency among the α-adrenoceptor agonists used in the present study, and the maximum response to guanfacine in the SRV was higher than that in the SRA (FIG. 6). Moreover, the maximum response to guanfacine in the SRV was equivalent to 45% of the maximum response to noradrenaline, whereas that in the SRA it was about 20% of the maximum response to noradrenaline.

The data demonstrates that compared to the full agonist noradrenaline, the maximum response to selective α₁-adrenoceptor agonist L-erythro methoxamine in the SRA and SRV accounted for only one-third of those to noradrenaline, thus indicating that L-erythro methoxamine probably acts as a partial α-adrenoceptor agonist in the sheep rectal vessels. Moreover, both sheep rectal vessels were much less sensitive to L-erythro methoxamine as compared to noradrenaline. However, L-erythro methoxamine was 3-fold arterioselective while noradrenaline was 4-fold venoselective in the sheep rectal vessels.

It is notable that guanfacine had the highest potency among the α-adrenoceptor agonists used in the present study of sheep isolated rectal arteries and veins, with pEC₅₀ values of 6.1 and 6.4, respectively. Moreover, the maximum response to guanfacine in the veins was slightly higher than the arteries.

### The Effect of Prazosin and RX811059 on Noradrenaline-Induced Vascular Contraction in the SRA and SRV

Prazosin is an α₁-adrenoceptor antagonist and RX811059 is an α₂-adrenoceptor antagonist. In the SRA, prazosin (0.4-10 nM) produced a concentration-dependent, parallel rightward shift of the CRC for noradrenaline (FIG. 7a). In contrast, RX811059 (30 nM) caused no change in the noradrenaline CRC (FIG. 7b). The addition of 30 nM RX811059 to 10 nM prazosin had no further effect on contraction induced by noradrenaline in the SRA (FIG. 7c). Based on the effect of 2 nM prazosin against noradrenaline-induced contraction, the pK_{B} value for the antagonist in the SRA was 9.40±0.17 (n=4).

In the SRV, prazosin (10 nM) caused a 70-fold rightward shift of the CRC for noradrenaline, with evidence of a small component resistant to prazosin (FIG. 8a). Meanwhile, RX811059 (30 nM) produced a parallel, 3-fold rightward shift of the CRC to noradrenaline without any change in the maximum response (FIG. 8b). The pEC₅₀ of noradrenaline in the absence of RX811059 was 6.41±0.29 (n=6) whereas that in the presence of RX811059 was 5.90±0.22 (n=6). The addition of 30 nM RX811059 to 10 nM prazosin produced a further 1.6-fold rightward shift of the CRC for noradrenaline. More importantly, responses to a low concentration of noradrenaline in the presence of prazosin were inhibited by the addition of RX811059 (FIG. 8c). Based on 10 nM prazosin and 30 nM RX811059, the pK_{B} values of prazosin and RX811059 in the SRV were 9.77±0.21 (n=13) and 7.86±0.13 (n=6), respectively.

To summarise in the SRA and SRV, contraction to noradrenaline was inhibited with higher affinity by prazosin (pK_{B}=9.4-9.8), suggesting the presence of α₁-adrenoceptors in both vessels. While RX811059 has no antagonising effect in the SRA, it produced a parallel, 3-fold rightward shift of the CRC to noradrenaline in the SRV with an approximate pK_{B} value of 7.9. At the same time, RX811059 inhibited the prazosin-insensitive contraction to noradrenaline in the veins. These observations indicate that noradrenaline mediates vascular contraction in the SRV via both α₁- and α₂-adrenoceptors, although the former makes the greater contribution.

### Which α-Adrenoceptor Subtype is Responsible for Guanfacine-Induced Vascular Contraction?

As previously shown in FIG. 6, guanfacine was more active in the vein, which has prazosin-resistant contractions to noradrenaline. This observation raised the question which α-adrenoceptor subtype is responsible for guanfacine-induced vascular contraction. Accordingly, the association of guanfacine and α₁-/α₂-adrenoceptors was evaluated using a paired segment design of the SRV. As shown in FIG. 9, guanfacine caused concentration-dependent contractions (Eₘₐₓ=54.7±8.1 and pEC₅₀=6.58±0.07, n=7). But, while 10 nM prazosin and 30 nM RX811059 appeared to reduce the maximum response, the combination was clearly more effective than either alone. In the presence of both antagonists, maximum response and pEC₅₀ of guanfacine were significantly reduced to 31.3±4.2 (P=0.025) and 6.04±0.19 (P=0.029), respectively—analysed by ANOVA with Dunnett's post hoc test.

Based on the experimental results, the effects of the individual α-adrenoceptor antagonists were variable, but the combination of both prazosin and RX8110159 clearly reduced contraction in the SRV—but did not abolish the response. Thus, both α-adrenoceptor subtypes are implicated in the contractile response to guanfacine, together with perhaps a non-adrenoceptor component.

### The Effect of Calcium Channel Activators and Flavonoids on the SRA and SRV

The dihydropyridine-type calcium channel activator S(−)-BayK8644 (1 μM) caused spontaneous contraction of the SRA in only 1 out of 18 observations (6%). In contrast, 1 μM S(−)-BayK8644 produced spontaneous, phasic contractions of the SRV and/or an increase in resting tension, in 31 out of 81 observations (38%) (FIG. 10). Meanwhile, there was no spontaneous contraction after the administration of diosmin (10 μM) or myricetin (10 μM) to the sheep isolated rectal vessels.

S(−)-BayK8644 significantly increased the potency of KCl in the SRA and noradrenaline in the SRV (FIG. 10). However, there was no effect of S(−)-BayK8644 on 5-HT-induced contraction in SRA and SRV.

The present studies revealed that while both α₁- and α₂-adrenoceptors are present in the sheep rectal vessels that connect to the equivalent of the haemorrhoid arteriovenous plexus, α₂-adrenoceptors only appear to contribute to contractile responses on the venous side.

Furthermore, the present studies revealed that: i) S(−)-BayK8644 (even at low concentration) enhanced KCl-induced contraction in the SRA and noradrenaline-induced contraction in the SRV; and ii) S(−)-BayK8644 induced spontaneous, phasic contraction and/or an increase in resting tension in 38% of the veins, but only 6% in the arteries. The increased contractile response to noradrenaline in the SRV by S(−)-BayK8644 appears to be the preferential effect of S(−)-BayK8644 on α₂-adrenoceptors.

### The Effect of S(−)-BayK8644 on Guanfacine-Induced Contraction of the SRV

As guanfacine mediates vascular contraction via the activation of both α1- and α₂-adrenoceptors and it is more potent in the veins (FIG. 3), if S(−)-BayK8644 can enhance the contractile response to guanfacine in vein then the combination of these two agents would be an effective drug formula for the treatment of haemorrhoids and other anorectal conditions. Using the SRV as an animal vascular model, S(−)-BayK8644 (1 μM) significantly increase the maximum response to guanfacine (37.0±6.6 vs 72.8±9.0; P=0.006, n=9) as shown by a non-parallel, concentration-dependent leftward shift of the CRC to guanfacine (20 nM-20 μM) in FIG. 11 Notably, S(−)-BayK8644 caused a non-significant increase in the potency of guanfacine. The pEC₅₀ values of guanfacine in the presence and absence of S(−)-BayK8644 were 6.64±0.16 and 6.48±0.11, respectively (P=0.41, n=9).

### Conclusion

The present data indicates that guanfacine had the highest potency among several α-adrenoceptor agonists used in the present study of SRA and SRV, and was involved as a vasoconstrictor. Furthermore, it demonstrates that S(−)-BayK8644, a calcium channel activator, preferentially enhanced vasoconstrictor responses to α-adrenoceptor agonists acting via α₂-adrenoceptors.

### 3. PRESENCE OF ALPHA-ADRENOCEPTORS IN HUMAN HAEMORRHOID TISSUE

### Materials and Methods

### Tissue Preparation

After obtaining approval from the local research ethics committees (Reference number: 05/Q2403/171) and having written consent from the patient, human haemorrhoid tissue was collected from patients with grade III or grade IV hemorrhoids who underwent haemorrhoidectomy at the Division of Gastrointestinal Surgery, Queen's Medical Centre, University of Nottingham, UK, between March 2008 and December 2008. For comparison, rectal mucosa and submucosa were obtained from rectal cancer patients who underwent anterior resection or abdominoperineal resection without previous pelvic radiotherapy. Meanwhile, the anal cushions were collected from patients undergoing total proctocolectomy for ulcerative colitis without anorectal involvement. In an operating room, the required anorectal tissues were dissected from the whole surgical specimen immediately after it was removed from the patient. The dissected specimens, usually less than 3 cm in size, were individually wrapped by a piece of aluminum foil. They were then placed on dry ice for 10-15 minutes to freeze the tissues. The packages of frozen tissue were then stored in an −80° C. freezer. For autoradiography the frozen archival tissue was sectioned at 6 μm using a rapid sectioning cryostat (Leica CM1900, Leica Microsystems Wetzlar GmbH, Germany) at −25° C. The sectioned tissue was immediately mounted on polylysine-coated adhesion slides (VWR International bvba, Germany), and stored at −80° C. until used.

In Vitro Autoradiograhy of α₁- and α₂-Adrenoceptors

Frozen slide-mounted tissue sections of 5 haemorrhoid specimens were removed from −80° C. storage and allowed to defrost at room temperature for 20 minutes. Slide-mounted sections of sheep anorectum, and rat brain were run in parallel and used for the validation of autoradiography of α₁- and α₂-adrenoceptor, respectively. All sections were pre-incubated in ice-cold 50 mM Tris HCl, pH 7.4, for 15 minutes at room temperature to remove endogenous ligand. Sections were then incubated in Tris HCl buffer containing 5 nM [³H]-prazosin (specific activity 3.22 TBq/mmol, GE Healthcare, UK) to identify α₁-adrenoceptor binding sites, with or without 100 μM unlabelled noradrenaline (Sigma Aldrich Chemie Gmbh, Germany), at 4° C. for 1 hour. Selected sections were incubated in Tris-HCl buffer containing 25 nM [³H]-RX821002 (specific activity 2.37 TBq/mmol, GE Healthcare, UK) to identify α₂-adrenoceptor binding sites, with or without 100 μM unlabelled rauwolscine (Carl Roth Gmbh, Germany), at 4° C. for 2 hours. Noradrenaline and rauwolscine were used to determine non-specific binding of α₁- and α₂-adrenoceptor ligands, respectively.

All assays were terminated by two 5-minute rinses in ice-cold TE buffer (50 mM Tris HCl, 1 mM EDTA, pH 7.4), followed by a dip into distilled water for removal of any remaining salt before transferring the sections into slide racks. They were then dried using a stream of warm air for 10-15 minutes and then cool air until completely dry. Slide-mounted sections were exposed to ³H-sensitive Hyperfilm™ (GE Healthcare, UK) in a light-tight x-ray cassette, with ³H microscale autoradiography standards (Amersham Bioscience, UK). The cassettes were then stored at 4° C. in light-proof conditions. Autoradiographs were generated at 1.5, 4, and 6 months after exposure to Hyperfilm™. Films were processed in accordance with the manufacturer's instructions.

Following the autoradiographic studies, selected sections were stained with haematoxylin and eosin (H&E) for histologic examination. First, sections were briefly washed with distilled water and then submerged in Harris Haematoxylin for 15-30 seconds. Next, they were washed in a bath of running tap water until the water cleared. Sections were then submerged in 0.1% acid alcohol for 30 seconds, following by washing in running tap water for 1 minute. Next, sections were placed in 0.1% Eosin for 2 minutes, followed by another 1-minute washing in running tap water. Sections were then dehydrated by 10-second dipping into ascending alcohol solutions (50%, 70%, 90%, and 100%×3). After that, they were submerged in xylene for 2-3 minutes in order to dissolve the remaining alcohol on the slide. Finally, DPX resin (a mixture of distyrene, a plasticiser, and xylene) was used to mount cover slips onto the tissue section. Sections were visualised under a microscope (Leica DM4000B, Leica Microsystems Wetzlar GmbH, Germany), and photographed where appropriate.

Binding was determined densitometrically using Bio spectrum AC Imaging System (Ultraviolet Products, Cambridge, UK). Sections were described as ‘total binding (TB)’ when incubated alone, whereas alternated sections were described as ‘non-specific binding (NSB)’ when incubated in the presence of excess subtype selective specific unlabelled ligand. In this case, the specific ligands for α₁- and α₂-adrenoceptor were noradrenaline and rauwolscine, respectively. The specific binding was determined by subtracting NSB from TB. The density of binding was presented as disintegrations per minute (dpm) radioactivity per mm² tissue (dpm/mm²).

Regarding the technique of density measurement, the largest square area of each section excluding the mucosa area was selected for the assessment of binding sites. The reasons for exclusion of mucosa area were: first, the study aimed to characterise receptors on blood vessels in haemorrhoids which were located in the submucosa, not in the mucosa. Second, haemorrhoids may be covered with different types of epithelium: squamous, columnar, or both. And, last, not all sections in this study contained mucosa. The measurement was performed twice.

### Results

In human haemorrhoid tissue the presence of α₁- and α₂-adrenoceptors was quantified by in vitro autoradiography using the α₁-adrenoceptor antagonist [³H]-prazosin and the α₂-adrenoceptor antagonist [³H]-RX821002, respectively. The most striking observations from the autoradiography studies were that the density of α₂-adrenoceptor binding was approximately 2-times higher than α₁-adrenoceptor binding in both mucosal and submucosal area of haemorrhoids, and the density of α₁- and α₂-adrenoceptors binding in the mucosa was about two-fold greater than that in the submucosa.

### 4. EXAMINATION OF THE CHARACTERISTICS OF ALPHA-ADRENOCEPTORS IN HUMAN ISOLATED MESENTERIC (COLONIC OR RECTAL) ARTERY AND VEIN

### Materials and Methods

### Wire Myography

After obtaining approval from the local research ethics committee (Reference number: 05/Q2403/171) and having written consent from the patient, human mesenteric (colonic or rectal) vessels were collected from patients who underwent colectomy and/or proctectomy at the Division of Gastrointestinal Surgery, Queen's Medical Centre, University of Nottingham, UK, between August 2009 and March 2010. Patients with a history of previous abdominal/pelvic radiotherapy, and patients with endocrine tumours or concomitant intraabdominal infection were excluded from this study. In an operating room, the mesentery containing the required segment of blood vessels was dissected from the whole surgical specimen immediately after it was removed from a patient. The tissue was then kept in a 100-ml pot containing pre-oxygenated Krebs-Henseleit buffer solution maintained at 4° C. until used. No experiments were conducted on tissue stored for more than 12 hours after dissection. The Krebs solution consisted of 118.4 mM NaCl, 4.7 mM KCl, 1.2 mM MgSO₄, 1.2 mM KH₂PO₄, 1.25 mM CaCl₂, 24.9 mM NaHCO₃, and 11.1 mM glucose.

Before setting a wire myography experiment, human mesenteric (colonic/rectal) vessels were meticulously dissected from the surrounding mesenteric fatty tissue. The ‘clean’ vessel was then divided into 5-mm ring segments. Generally, the internal diameter of vessels ranged between 400-1000 μm for human mesenteric artery (HMA) and 500-1200 μm for human mesenteric vein (HMV). Each 5-mm ring segment was suspended between two 200-μm wire supports in an organ bath containing 20 ml Krebs-Henseleit buffer solution. The contractile study of human mesenteric vessels was then set in the similar manner of the previous study on sheep isolated rectal vessels.

After a 20-minute equilibration period, an initial resting tension of 8 and 1 gram weight (g wt) was gradually applied to arterial and venous ring segments, respectively, and left to relax for 30 minutes. The final resting tension varied between 1.8 g wt and 3.2 g wt for HMA, and between 0.07 g wt and 0.35 g wt for HMV. Once the vascular segments were fully settled, they were tested for contractile activity with 60 mM KCl three occasions, and an agonist concentration-response curves (CRC) was then reconstructed after the vessels were left to equilibrate for 60 minutes as previously described for sheep rectal vessels. Notably, the maximum contraction (tension) produced by the third-time administration of 60 mM KCl was used as a reference contraction to which subsequent responses were compared. In the case of noradrenaline study, 10 μM cocaine was added into the organ bath 40 minutes prior to the administration of noradrenaline for inhibiting the uptake of noradrenaline by neural tissue within the vascular wall (re-uptake 1) (Westfall & Westfall, 2006 In Goodman & Gilman's the Pharmacological Basis of Therapeutics. eds Brunton, L. L., Lazo, J. S. & Paker, K. L. pp. 137-181. New York: McGraw Hill).

Although it appeared that the CRCs of several vasoconstrictors were reproducible (no significant change in maximum response and agonist potency) in human mesenteric vessels, a single CRC, paired segment approach was adopted throughout the study of human blood vessels. This was due to a desire to limit the time of tissue storage and the ease of tissue preparation. The single CRC, paired segment approach involves the generation of only one agonist CRC per tissue. The maximum response (Eₘₐₓ) and pEC₅₀ (defined as the negative logarithm of the concentration required to produce 50% of the maximum response) of each vascular segment were compared to those of other vascular segments (adjacent segments from the same patient, or other segments from different patients).

Similar to the contractile studies of sheep rectal vessels, if the response exhibited either phasic or non-sustained characteristics, the highest point of the contractile tension was used as the maximum response of that agonist concentration. If the vessel had spontaneous and periodic contraction after the administration of S(−)-BayK8644—which occurred in 70% of HMA and 80% of HMV, the effect of agonist would be considered when there was an increase in the frequency of contraction as well as a rise in the baseline of vascular tone.

The selective α₁-adrenoceptor antagonist prazosin and selective α₂-adrenoceptor antagonist RX811059 were used in this study. The α-adrenoceptor antagonists and Ca²⁺ channel activator S(−)-BayK8644 were added into the organ bath 40-50 minutes prior to the administration of agonists.

Data and statistical analysis, and the drugs, were as described previously.

### Results

The experiments undertaken and described below used human isolated mesenteric artery and vein tissue to demonstrate that functional α₁-adrenoceptors mediating vascular contractions are expressed in both human isolated mesenteric artery (HMA) and vein (HMV). Furthermore, functional α₂-adrenoceptors mediating vascular contractions are mainly present in the HMV. The experiments demonstrate that the calcium channel activator S(−)-BayK8644 enhances the vasoconstrictor effect of several agents including noradrenaline and guanfacine in both vessels.

In the HMV, 10 nM prazosin and 30 nM RX811059 significantly reduced the potency of noradrenaline and both antagonists in combination were clearly more effective than either alone, confirming the presence of both α₁- and α₂-adrenoceptors mediating contraction in the vein. In contrast, noradrenaline-induced contractions of the HMA were inhibited only by prazosin (pK_{B} 9.50), but not by 30 nM RX811059, suggesting that there was only α₁-adrenoceptors mediating vasoconstriction in the artery.

Guanfacine was demonstrated, like in the sheep vessels, to cause contraction of the human vessels as summarised in FIG. 12.

FIG. 13 demonstrates that guanfacine caused concentration-dependent contractions (Eₘₐₓ=31.4±4.1 and pEC₅₀=5.65±0.06, n=6). While 10 nM prazosin appeared to reduce the maximum response without a change in drug potency (Eₘₐₓ=18.4±4.1; P=0.65 and pEC₅₀=5.61±0.10; P=1.00), 30 nM RX811059 caused a 2-fold parallel rightward shift of CRC to guanfacine (Eₘₐₓ=33.0±4.1; P=1.00 and pEC₅₀=5.28±0.09; P=0.14). Meanwhile, the combination of both antagonists seemed to be more effective than either alone. In the presence of both antagonists, Eₘₐₓ and pEC₅₀ values of guanfacine were 20.3±8.5 and 5.19±0.15, respectively. These findings suggest that both α₁ and α₂-adrenoceptors are implicated in the contractile response to guanfacine.

### The Effect of Calcium Channel Activator S(−)-BayK8644 on the HMA and HMV

Previous studies of the sheep rectal vessels demonstrated that S(−)-BayK8644 increased the contractile response to some α-adrenoceptor agonists. The effect of S(−)-BayK8644 was explored in the HMA and HMV on the basis of contractile responses to KCl and various selective and non-selective α-adrenoceptor agonists.

The dihydropyridine-type calcium channel activator S(−)-BayK8644 (1 μM) caused spontaneous phasic contraction, with or without an increase in resting tension, in 5 out of 7 HMA (71%) and 15 out of 18 HMV (83%). S(−)-BayK8644 tended to increase either agonist potency, maximum response, or both potency and response to KCl and the α-adrenoceptor agonists used in this study (FIG. 14).

### 5. α1-ADRENOCEPTOR VS α2-ADRENOCEPTOR LIGANDS

It has been shown that α2-adrenoceptors are better able than α1-adrenoceptors to oppose the vasodilator action of agents capable of raising cyclic AMP (Roberts et al (1998) Br. J Pharmacol. 124, 107-114; Roberts et al (1999) Br. J Pharmacol. 128, 1705-1712). The α2-adrenoceptor subtype in vascular smooth muscle is negatively coupled to cyclic AMP and, in contrast to α1-adrenoceptors, directly opposes the effect of this cyclic nucleotide. Observations in the porcine splenic artery, palmar lateral vein, digital artery and tail artery (FIG. 20) demonstrate that responses to selective α2-adrenoceptor agonists are relatively insensitive to the vasodilator actions of forskolin—a vasodilator that stimulates adenylyl cyclase. FIG. 20 shows that contractile responses to brimonidine (UK-14034), unlike those to phenylephrine (an α1-adrenoceptor agonist) are enhanced in the presence of the combination of U46619 and forskolin. Since the sensory neuropeptides calcitonin gene-related peptide (CGRP) and vasoactive intestinal polypeptide (VIP) mediate a vasodilator effect by stimulating adenylyl cyclase, the increase in blood flow (and anal cushion volume) mediated by these agents would be effectively opposed by an α2-adrenoceptor agonist. This observation favours the use of α2-adrenoceptors ligands to reduce the anal cushion ‘hypertension’ in patients with haemorrhoids.

### 6. ANATOMICAL CONSIDERATIONS

The anal cushions associated with the sphincter are highly vascularised structures that primarily contribute to sphincter pressure and, therefore, to continence. FIG. 15 shows a microcast of a human anal cushion and illustrates that the structure comprises numerous arterio-venous anastomosis with no discernible capillary bed—the absence of the latter indicates that this structure does not have a major role in the exchange of gases and solutes. In this respect the vasculature of the anal cushions share the same volume-related function as corpus cavenosum of the penis. FIG. 15 also shows that in terms of overall volume of the structure the arterial side makes little contribution to the capacitance of the structure. Furthermore, since the venous side of the anal cushions possesses less smooth muscle it will not offer much resistance to increases in blood flow and will simply increase in volume in response to an elevation in blood flow. FIG. 16 shows immunohistochemical evidence that part of the venous side is associated with narrowing of the structure, which in turn appears to linked to higher density of contractile proteins. This type of anatomical arrangement is consistent with a sphincter-like structure that would tend to limit of the overall capacitance of the anal cushion. Such sphincter-like structures could also participate in rhythmic contractions to move blood out of the cushions and prevent pooling. In this respect, it is noteworthy that rhythmic contractions were noted in sheep veins, but not arteries, and these were greatly enhanced by the calcium channel agonist Bay K-8644. Thus, the combination of a calcium channel agonist with a venoselective α₂-adrenoceptor agonist is likely to reduce overall volume and offset the excessive swelling associated with the development of haemorrhoids. These anatomical considerations also help to explain why an arterio-selective approach to reducing blood flow into the plexus, (as in the case of Preparation H which contains the selective α₁-adrenoceptor agonist phenylephrine), may reduce the rate of ‘filling’ of the venous side of the structure but have little impact on the overall size of the haemorrhoid because the rate of emptying and capacitance is unchanged.

### 7. INCREASED NITRIC OXIDE LEVELS

Western blot analysis of human haemorrhoidal tissue reveals the presence of all three isoforms of nitric oxide synthase, each of which are present in greater quantity than in the allied rectal submucosal tissue (FIG. 4.15 of Lohsiriwat 2011). Furthermore, immunohistochemical analysis reveals that all three isoforms of nitric oxide synthase are closely associated with both nerves and the endothelium in haemorrhoidal tissue (see FIGS. 4.9-4.12 Lohsiriwat 2011 PhD thesis, and reproduced herein as FIG. 19).

The presence of elevated levels of nitric oxide in the anal cushions appears likely because of the release of substance P from sensory nerves and also the increased arterial blood flow contributing to greater shear stress on the endothelium. Therefore by targeting the presence of nitric oxide anorectal conditions such as haemorrhoids, piles, anal fissures and anorectal vascular malformations may be treated.

Although there are various synthetic inhibitors of this enzyme (eg. N(G)-L-arginine methyl ester—L-NAME), a number of endogenous substances are also capable of inhibiting the enzyme at high concentrations. Asymmetric dimethyl arginine (ADMA) can be found in the plasma at submicromolar concentrations (approx 0.5 μM), but at much higher concentrations (100 μM) has been reported to inhibit nitric oxide synthase (Bayliss (2006) Nat. Clin. Pract. Nephrol. 2, 209-220). In addition, in some vascular preparations the presence of 100 μM ADMA has been shown to enhance the vasoconstrictor responses to phenylephrine, presumably by removing the influence of basal nitric oxide (Al-Zobaidy et al (2010) Br. J. Pharmacol. 160, 1475-1483). Thus a combination of a selective α2-adrenoceptor agonist and ADMA would reduce the volume of anal cushions (by a preferential vasoconstrictor action on the venous side of the arterio-venous plexus) and lower blood flow by opposing the dilator response to nitric oxide.

### 8. DISCUSSION

The observation, supported by the data presented herein, that there is a higher density of α₂-adrenoceptor than α₁-adrenoceptor in the sheep anorectal mucosa and rectal vessels and the ability of these adrenoceptors to by stimulated by various ligands, and in particular ligands which are agonists of the α₂-adrenoceptor, demonstrates that compositions that target the α₂-adrenoceptor would allow the treatment of haemorrhoids or other anorectal conditions in man.

Interestingly, the pharmacological characteristics of α-adrenoceptors in the sheep isolated rectal artery (SRA) and vein (SRV) were comparable to those in the human isolated mesenteric (colonic/rectal) artery (HMA) and vein (HMV). The SRV and HMV had functional α₁- and α₂-adrenoceptors mediating vascular contraction, while the SRA and HMA expressed only vasoconstricting α₁-adrenoceptors, with a possibility of minimal distribution of vasoconstricting α₂-adrenoceptors in the HMA.

The data presented herein shows that human anorectal tissue and the haemorrhoid arteriovenous plexus in particular contains various vascular receptors, e.g. α₁-/α₂-adrenoceptors, which could be targeted for pharmacological modulation of vascular tone of these haemorrhoidal vessels.

In particular the data presented demonstrates that agonists of α₂-adrenoceptors, such as guanfacine, could be applied as a topical treatment of haemorrhoids because i) it mediates vascular contraction through α-adrenoceptors (preferentially via α₂-adrenoceptors), ii) it has high affinity to venous channels which are the major component of haemorrhoids, iii) it has been clinically used in man with minimal side effects, and iv) the vasoconstrictor effect of guanfacine still remained even in the direct application.

Furthermore, agents that could potentiate the vasoconstrictor effect of α-adrenoceptors agonists were also identified, for example the dihydropyridine-based calcium channel activator S(−)-BayK8644 is capable of increasing the potency and/or contractile responses to various α-adrenoceptor agonists.

1. A composition comprising an α-adrenoceptor ligand for use in the treatment of benign anorectal conditions.

2. The composition of claim 1 wherein the ligand is an α₁ and/or an α₂-adrenoceptor ligand.

3. The composition of claim 1 or claim 2 wherein the ligand is an α₂-adrenoceptor ligand.

4. The composition of claim 3 wherein the α₂-adrenoceptor ligand is an agonist of the α₂-adrenoceptor.

5. The composition of claim 4 wherein the agonist of the α₂-adrenoceptor is selected from the group comprising apraclonidine, brimonidine, clonidine, detomidine, dexmedetomidine, guanabenz, guanfacine, lofexidine, medetomidine, romifidine, tizanidine, tolonidine, xylazine, fadolmidine, xylometazoline and oxymetazoline or pharmaceutically active salts, esters, amides or N-oxides thereof.

6. The composition of any preceding claim wherein the ligand is guanfacine or a pharmaceutically active salt, ester, amide or N-oxide thereof.

7. The composition of claim 3 wherein the α₂-adrenoceptor ligand is an antagonist of the α₂-adrenoceptor.

8. The composition of claim 7 wherein the antagonist of the α₂-adrenoceptor is selected from the group comprising atipamezole, cirazoline, efaroxan, idazoxan, mianserin, mirtazapine, napitane, phenoxybenzamine, phentolamine, rauwolscine, setiptiline, tolazoline and yohimbine or pharmaceutically active salts, esters, amides or N-oxides thereof.

9. The composition of any preceding claim further comprising more than one α-adrenoceptor ligand.

10. The composition of any preceding claim further comprising an inhibitor of nitric oxide synthase

11. The composition of any preceding claim further comprising a calcium channel activator.

12. The composition of claim 11 wherein the calcium channel activator is a dihydropyridine-based calcium channel activator or a pharmaceutically active salt, ester, amide or N-oxide thereof.

13. The composition of claim 11 or 12 comprising guanfacine or a pharmaceutically active salt, ester, amide or N-oxide thereof and S(−)-BayK8644 or a pharmaceutically active salt, ester, amide or N-oxide thereof.

14. The composition of any preceding claim further comprising one or more a steroid or a pharmacologically acceptable derivative thereof, an analgesic agent, an antimicrobial agent, an antiviral agent, an antifungal agent, an anti-inflammatory agent and an antidiarrheal agent.

15. The composition of any preceding claim wherein the benign anorectal condition is one or more of haemorrhoids, piles (the pathological condition of haemorrhoids), anal fissures, post operative treatment following haemorrhoidectomy, anal symptoms following vaginal delivery (with or without episiotmy), and anorectal vascular malformations.

16. The composition of any preceding claim wherein the composition is intended for topical administration.

17. The composition of any preceding claim comprising between 0.03% and 0.1% by weight of an α adrenoceptor ligand, or which is intended for use at a concentration of between 0.03% and 0.01% by weight of α adrenoceptor.

18. The use of an α-adrenoceptor ligand, or a pharmaceutically active salt, ester, amide or N-oxide thereof, in the manufacture of a medicament for the treatment of an anorectal condition.

19. The use of claim 18 wherein the α-adrenoceptor ligand is an α₂-adrenoceptor ligand.

20. A topically acting pharmaceutical composition comprising an α-adrenoceptor ligand or a pharmaceutically active salt, ester, amide or N-oxide thereof, and a pharmaceutically acceptable carrier.

21. The composition of claim 20 wherein the α-adrenoceptor ligand is an α₂-adrenoceptor ligand.

22. The composition of claim 21 further comprising an inhibitor of nitric oxide synthase.

23. A method of treatment of a benign anorectal condition in a subject comprising administering to the subject an effective amount of a composition according to any of claim 1 to 16, 20, 21 or 22.
